SEQUENCE LISTING (20 sequences)

SEQ ID NO: 1 - protein, 549 aa, Rattus norvegicus
MTRAAERGQGATGWGLRGALMAVALLSVLNAVGTVFVLYQWRELSAALRALEAQHGQEQREDSALRAFLAELSRAPARVPEPPQDPMSAARNKRSHGGEPASHIRAESQDMMMMMTYSMVPIRVMIDLCNSTQGICLTGPPGPPGPPGAGGLPGHNGSDGQPGLQGPKGEKGAVGKRGKMGLPGATGNPGEKGEKGDAGELGLPGNEGPPGQKGDKGDKGDVSNDVLLTGAKGDQGPPGPPGPPGPPGPPGSRRAKGPRQPNSFTNQCPGETCVIPNDDTLVGRADEKVNERHSPQTEPMITSIGNPAQVLKVKETFGTWLRESANRSDDRIWVTEHFSGIMVKEFEDLPALLNSSFTLLHLPHYFHGCGHAVYNNSLYYHKGGSNTIVRFEFGKETPQTLKLEDALYFDRKYLFANSKTYFNIAVDEKGLWIIYASSVDGSSILVAQLDERTFSVLRHINTTYPKSKAGNAFIAQGILYVTDTKDTRVTFAFDLLRGKQINANFGLRMSQSVLAMLSYNMRDQHLYSWEDGHLMLYPVHFSSTAPSQR

SEQ ID NO: 2 - skipped

SEQ ID NO: 3 - DNA, 19 bp, Artificial sequence
  siRNA
tgagcgccattctccacaa

SEQ ID NO: 4 - DNA, 19 bp, Artificial sequence
  siRNA
acaactctctctactacca

SEQ ID NO: 5 - skipped

SEQ ID NO: 6 - protein, 13 aa, Artificial sequence
  Synthetic peptidescorresponding to amino acid residues 273-287 present in the extracellular region of rat gliomedin
CVIPNDDTLVGRA

SEQ ID NO: 7 - protein, 23 aa, Rattus norvegicus
LRGALMAVALLSVLNAVGTVFVL

SEQ ID NO: 8 - protein, 123 aa, Rattus norvegicus
VMIDLCNSTQGICLTGPPGPPGPPGAGGLPGHNGSDGQPGLQGPKGEKGAVGKRGKMGLPGATGNPGEKGEKGDAGELGLPGNEGPPGQKGDKGDKGDVGAKGDQGPPGPPGPPGPPGPPGSR

SEQ ID NO: 9 - protein, 245 aa, Rattus norvegicus
PMITSIGNPAQVLKVKETFGTWLRESANRSDDRIWVTEHFSGIMVKEFEDLPALLNSSFTLLHLPHYFHGCGHAVYNNSLYYHKGGSNTIVRFEFGKETPQTLKLEDALYFDRKYLFANSKTYFNIAVDEKGLWIIYASSVDGSSILVAQLDERTFSVLRHINTTYPKSKAGNAFIAQGILYVTDTKDTRVTFAFDLLRGKQINANFGLRMSQSVLAMLSYNMRDQHLYSWEDGHLMLYPVHFSS

SEQ ID NO: 10 - protein, 511 aa, Rattus norvegicus
YQWRELSAALRALEAQHGREQREDSALRAFLAELSRAPARVPEPPQDPMSAARNKRSHGGEPASHIRAESQDMMMMMTYSMVPIRVMIDLCNSTQGICLTGPPGPPGPPGAGGLPGHNGSDGQPGLQGPKGEKGAVGKRGKMGLPGATGNPGEKGEKGDAGELGLPGNEGPPGQKGDKGDKGDVSNDVLLTGAKGDQGPPGPPGPPGPPGPPGSRRAKGPRQPNSFTNQCPGETCVIPNDDTLVGRADEKVNERHSPQTEPMITSIGNPAQVLKVKETFGTWLRESANRSDDRIWVTEHFSGIMVKEFEDLPALLNSSFTLLHLPHYFHGCGHAVYNNSLYYHKGGSNTIVRFEFGKETPQTLKLEDALYFDRKYLFANSKTYFNIAVDEKGLWIIYASSVDGSSILVAQLDERTFSVLQHINTTYPKSKAGNAFIAQGILYVTDTKDTRVTFAFDLLRGKQINANFGLRMSQSVLAMLSYNMRDQHLYSWEDGHLMLYPVHFSSTAPSQR

SEQ ID NO: 11 - DNA, 1536 bp, Rattus norvegicus
taccagtggcgcgagctgagcgcggcgctgcgggcactggaggcgcaacacggccgggagcagcgcgaggacagcgccctacgcgcctttctagctgaattaagtcgtgcgccagcccgagtccccgaaccaccccaggaccccatgagtgcagcgcgcaataagcgcagccacggcggcgagcctgcgtcacacatccgcgccgagagccaggacatgatgatgatgatgacctactccatggtgccgatccgggtgatgatagacctgtgcaacagcacccagggcatctgccttacaggaccaccgggcccaccaggacctccaggagctggtgggttaccaggccacaatggatcagatggacagcctggtctccagggcccaaaaggagaaaaaggagcagttgggaagagaggaaaaatggggttacccggagccacaggaaatccaggggaaaagggagagaagggagatgctggtgaactgggcctacctggaaatgagggaccaccaggacagaaaggagacaaaggagacaaaggagatgtgtccaatgacgtgcttttgacaggtgccaaaggtgaccaagggccccctggcccacctggacccccagggcctccaggccctcctggaagcagaagagccaaaggccctcggcagccaaattcgttcaccaaccagtgtccaggggagacgtgtgtcatacccaatgatgataccttggtggggagagctgatgagaaagtcaatgagcgccattctccacaaacagaacccatgatcacgtccattggtaacccggcccaagtcctcaaagtgaaagagacttttgggacctggctaagagagtctgctaacaggagtgatgaccgcatttgggtgactgaacatttttcaggcatcatggtgaaggagtttgaagacctgcccgccctcctgaatagcagcttcaccctcctccacctcccacattacttccatggctgcgggcacgctgtttacaacaactctctctactaccacaaaggaggctccaacaccatagtgagatttgaatttgggaaagagacacctcaaactctgaagcttgaagatgctttgtattttgatcgaaaatacctctttgcaaattccaagacttacttcaacatagcagtggatgagaagggcctctggattatctacgcctcgagtgtggatggctcaagcatccttgtggcacagctggacgagaggacattctctgtgctgcagcacatcaataccacataccccaagtccaaggctggcaatgccttcatagctcaagggatcctctatgtcacggacaccaaagatacaagggtcacgtttgcctttgatttgttacgagggaagcagatcaatgcaaacttcggtctcagaatgtcacagtctgttcttgccatgttgtcatacaatatgagagaccagcatttgtactcgtgggaagacggccacctgatgctctatcctgtgcacttttcgtcaacagcacccagccagcgatag

SEQ ID NO: 12 - DNA, 879 bp, Rattus norvegicus
ctgggcctacctggaaatgagggaccaccaggacagaaaggagacaaaggagacaaaggagatgtgtccaatgacgtgcttttgacaggtgccaaaggtgaccaagggccccctggcccacctggacccccagggcctccaggccctcctggaagcagaagagccaaaggccctcggcagccaaattcgttcaccaaccagtgtccaggggagacgtgtgtcatacccaatgatgataccttggtggggagagctgatgagaaagtcaatgagcgccattctccacaaacagaacccatgatcacgtccattggtaacccggcccaagtcctcaaagtgaaagagacttttgggacctggctaagagagtctgctaacaggagtgatgaccgcatttgggtgactgaacatttttcaggcatcatggtgaaggagtttgaagacctgcccgccctcctgaatagcagcttcaccctcctccacctcccacattacttccatggctgcgggcacgctgtttacaacaactctctctactaccacaaaggaggctccaacaccatagtgagatttgaatttgggaaagagacacctcaaactctgaagcttgaagatgctttgtattttgatcgaaaatacctctttgcaaattccaagacttacttcaacatagcagtggatgagaagggcctctggattatctacgcctcgagtgtggatggctcaagcatccttgtggcacagctggacgagaggacattctctgtgctgcagcacatcaataccacataccccaagtccaaggctggcaatgccttcatagctcaagggatcctctatgtcacggacaccaaagatacaagggtcacgtttgccttt

SEQ ID NO: 13 - DNA, 174 bp, Rattus norvegicus
acaggaccaccgggcccaccaggacctccaggagctggtgggttaccaggccacaatggatcagatggacagcctggtctccagggcccaaaaggagaaaaaggagcagttgggaagagaggaaaaatggggttacccggagccacaggaaatccaggggaaaagggagagaag

SEQ ID NO: 14 - protein, 551 aa, Homo sapiens
MARGAEGGRGDAGWGLRGALAAVALLSALNAAGTVFALCQWRGLSSALRALEAQRGREQREDSALRSFLAELSRAPRGASAPPQDPASSARNKRSHSGEPAPHIRAESHDMLMMMTYSMVPIRVMVDLCNSTKGICLTGPSGPPGPPGAGGLPGHNGLDGQPGPQGPKGEKGANGKRGKMGIPGAAGNPGERGEKGDHGELGLQGNEGPPGQKGEKGDKGDVSNDVLLAGAKGDQGPPGPPGPPGPPGPPGPPGSRRAKGPRQPSMFNGQCPGETCAIPNDDTLVGKADEKASEHHSPQAESMITSIGNPVQVLKVTETFGTWIRESANKSDDRIWVTEHFSGIMVKEFKDQPSLLNGSYTFIHLPYYFHGCGHVVYNNSLYYHKGGSNTLVRFEFGQETSQTLKLENALYFDRKYLFANSKTYFNLAVDEKGLWIIYASSVDGSSILVAQLDERTFSVVQHVNTTYPKSKAGNAFIARGILYVTDTKDMRVTFAFDLLGGKQINANFDLRTSQSVLAMLAYNMRDQHLYSWEDGHLMLYPVQFLSTTLNQ

SEQ ID NO: 15 - DNA, 5084 bp, Homo sapiens
ctctgggctgctcgggcgccaccactactgtccccatcccgccccgccagctgggccaaagggtgacttgactcacgacgctgccaccagcccacggcttgcccgaggcgtataaaggctgccagggccgcaggctgccaagccctgccctgcccaaggcgcatagagcatggcccgaggcgctgagggaggccgtggggacgcgggttggggcctgcgtggcgccctggcggccgtggcgctgctctcggcgctcaacgctgcgggcacggtgttcgcgctgtgccagtggcgcgggctgagctcggcgctgcgggctttggaggcgcagcggggccgggagcagcgcgaggacagtgccctgcgctccttcctggccgagttgagccgcgcgccgcgcggggcgtccgcaccaccccaagacccggccagctcagctcgcaacaagcgcagccacagcggcgagcccgcgccgcatatccgcgccgagagccatgacatgctgatgatgatgacctactccatggtgccgatccgagtgatggtggacctgtgcaacagcaccaagggcatctgcctcacaggaccttctggaccaccaggacctccgggagccggcgggttgccaggacacaacggattggatggacagcctggtcctcagggcccaaaaggagaaaaaggagcaaatggaaaaagaggaaaaatggggatacctggagctgcaggaaatccaggggaaaggggagaaaagggagaccatggtgaactgggcctgcagggaaatgagggcccaccagggcagaagggagaaaagggtgacaaaggagatgtgtccaacgacgtgctcctggcaggtgccaaaggtgaccaaggcccacccggtccacctgggcccccaggccctccaggtcctccagggccccctggaagcagaagagccaaaggccctcggcagccaagcatgttcaacggccagtgcccaggtgagacttgtgccataccaaatgatgataccttggttggaaaagctgatgagaaagccagtgaacaccattccccacaagcagaatccatgatcacttccattggaaacccagtgcaagtactgaaagtgacagagacatttgggacttggataagagagtctgctaacaagagtgatgaccggatttgggtgacagagcatttttcaggcatcatggttaaggaattcaaggatcagccctcacttctgaatggcagttacacgttcatccaccttccatactatttccatggctgtgggcacgttgtttacaacaactctctctactaccacaaagggggttctaataccctagtgagatttgaatttggccaggaaacatcccaaactctgaagcttgaaaatgccttgtattttgatcgaaaatacctttttgcaaattccaaaacttacttcaatctagctgtagatgaaaagggcctttggattatctatgcgtcaagtgtggacggctcgagcattcttgtagcacaactggatgagaggacattctcagtggtgcaacacgtcaataccacgtaccctaaatccaaggctggcaacgccttcattgcccgaggaatcctctatgtcacagacaccaaagatatgagggtcacatttgcctttgatttgttaggagggaaacagatcaatgcaaactttgatttaagaacttcccagtctgttcttgccatgttagcatacaacatgagagatcagcatttatattcatgggaagatggccatttaatgctttatcctgtgcagtttttgtcaactaccttaaatcagtgatgtgctgcattcggctcccttcagcaaatttcaggggttttctgggaccagttctcccccaacaggaaacttgtttttttaacgtcagccagatatttagaaaataacctcaaaagtgtttatatggtcagtgagccccgcttagtgaaatagcaacagattggaagttgaaatggctgagatttggtgatctccccacagctggctctgcaagttacctctttctccttgggccttagtttccccattggtaatctgaattggctaagatgattggggagattttctgtacctgtaggtaatttggtgattcttggtggctgctcttctcacaacttttatgtatctgcttctgtcgtttagcttttttagccacatgctgaccaaatttacctttgagttgataagtccagtggcttgagtagtgaatccctcagtgctgacttatatcttgttctttgaaaaaatgcattgactctttaagacatctaaagtatcacattatccataatttattgcttttctttgcatctgcacctgccaccacagaataaccattaccctcagctgctgattgggcagctctgagattagcaaaagccagggacagctacatgttcaggtttttttttttttttttttttcaataagctattttttttcttttcttattttaaatagagagagagtcttgctatgtttcccaggctggtcttgaactcctggggctcaagtgatcctcctgccttggcctcccaaaatgctggattacaggcatgtgtgcctggcccaggtttcttaataaaacagaatcatgatcttccaggttccccccagtttctgatcatgttgatttgtagctgtggatcatgaacactgaatcctcagatcactctgacttcttatgcttctcctgtggatccactatcaaagtactaaatgctgtgtaagtagacgttaatctggctggaaccatgggaagcactttgcagtgttcagaagagaggctccatttgtggctattatgtagaactgggccagagccagtccattgcctgtttttttaaataaggttttactgagcacagccacactcatttgtttatgcagtacggcctgacattgcttttgctctgcaacagcagagtcgagtcattgcaacaaagagcatatggccccacagtgcctaaaatattgaccagctacccctttatggaaaaagattgctgactcctgataaagaatataaagtgagcctgattcttgaaaaaatcagaaccagagcctgttttgttttgttctaaactaagaagccgcataggatgtgacttgcgttttgagtagaggggaaggctgataacggcgtaagatgaagtggccctccacaaaggctggttaggggacagttctttctctaacatagttttaaaggatgtgatctggtccccttggatgccaggagagaatccagttgaacttgctcctaaatgctcttaaatatgcatattttctgccaactcacttctttaaacatctttcagcccagcgctgcggccccgggaagggccactgcgaatagagaggaagctggaaaagttcctggggctctgcagccaggaaggggaaccagggcaaatcttatgtaaagatttttcagcaacttgtcccaatttgtgtgtattctgaaactttctctttgggaccaaattcattctcaatggccctgagttcaatatattattaacagcagtattttaaaacttagggttgaactgggcatggtggcacataactgcaatcccagctactttggaggcagggatgggaggatcacttgaggccaggatctcaggaccagcctagagagatcccatctctaaaaaataaaatataagaaaataaaacttaggggatatacagatttaaatattcaaatctccctgctcccctgaaagtccccaggcagctgtcaatgacttgtttgttgtgttctcaatatgatggctatttgaaacttcacctacttttcattagattggttgtaccatgtcaccttagcttttaaaaatactcttttcagattcacgttctctaacaaagagtctcatgttcaagatcaatatgtctaataagtgctggtgtccttttaaagtatttaaatatatatgttgctgttgctgaatacaggagaccaggttaggaatatagtttcataataatagtacatacaatactaattgtatataaggtagcaaccaaaagaggttgttaattagcacatattccttttagaaaaatgtttcagaaacctcagtcttgatatctgagctatctgggctcccttacttgtgagtaagggatcatgctcaccactggagaagcttacaccgggactttttttcttttttcttttttttttgctatgacagagtaatgctaacgtaaggacaactgagtttgatcagtgtttaatcgcagtgggtaatcttatctgattgtctttaaaagtgaaaaggattaagattttattctttcttgtaaacattacttgattttttaaagaagttttgggctcactgctaaaatagagtatacaactgaatgtttttaagtcaagatactgttttaggagtttaccctctcatttataaccaaagttgctctaaaacactttccaaatatctgcacttctgatgtcagaatcaaaccagataattctctaattcttctttaatctaaagtagatagcttcccactggaaagtaaacaaaaccatccctcccaacctcaaagctaggccacactctatttcaaggcattttctttcagctgataaggtgtcctcctgaagccaagtaggtggttctggtctccaagtatcgttaagcacaggtgctatgacagaaaaagttctggggtggaagttttaagatgaggagttctgatcttaggcatcttaacagtcacaaggtgaaaagtcaaatgaaacagtacaattcttgatgagtgaggtgtcatcttccaaccacacagaggacgttttggctatgatcatctgatggcaagtgaaggagaaatgagtgatagggctttgcgttttcatccagatgctgtggccctgtgtttcacagcattaagagccataatttccaacctgcacagatcctgaacaacaaatgaataacgatgaatgtctttttggttgtaatttaacaagtcaaataaaataatcattgctgagcacaatcacatgttgaaa

SEQ ID NO: 16 - protein, 7 aa, Rattus norvegicus
SNDVLLT

SEQ ID NO: 17 - protein, 7 aa, Homo sapiens
SNDVLLA

SEQ ID NO: 18 - protein, 513 aa, Homo sapiens
CQWRGLSSALRALEAQRGREQREDSALRSFLAELSRAPRGASAPPQDPASSARNKRSHSGEPAPHIRAESHDMLMMMTYSMVPIRVMVDLCNSTKGICLTGPSGPPGPPGAGGLPGHNGLDGQPGPQGPKGEKGANGKRGKMGIPGAAGNPGERGEKGDHGELGLQGNEGPPGQKGEKGDKGDVSNDVLLAGAKGDQGPPGPPGPPGPPGPPGPPGSRRAKGPRQPSMFNGQCPGETCAIPNDDTLVGKADEKASEHHSPQAESMITSIGNPVQVLKVTETFGTWIRESANKSDDRIWVTEHFSGIMVKEFKDQPSLLNGSYTFIHLPYYFHGCGHVVYNNSLYYHKGGSNTLVRFEFGQETSQTLKLENALYFDRKYLFANSKTYFNLAVDEKGLWIIYASSVDGSSILVAQLDERTFSVVQHVNTTYPKSKAGNAFIARGILYVTDTKDMRVTFAFDLLGGKQINANFDLRTSQSVLAMLAYNMRDQHLYSWEDGHLMLYPVQFLSTTLNQ

SEQ ID NO: 19 - protein, 245 aa, Homo sapiens
SMITSIGNPVQVLKVTETFGTWIRESANKSDDRIWVTEHFSGIMVKEFKDQPSLLNGSYTFIHLPYYFHGCGHVVYNNSLYYHKGGSNTLVRFEFGQETSQTLKLENALYFDRKYLFANSKTYFNLAVDEKGLWIIYASSVDGSSILVAQLDERTFSVVQHVNTTYPKSKAGNAFIARGILYVTDTKDMRVTFAFDLLGGKQINANFDLRTSQSVLAMLAYNMRDQHLYSWEDGHLMLYPVQFLS

SEQ ID NO: 20 - protein, 119 aa, Homo sapiens
TGPSGPPGPPGAGGLPGHNGLDGQPGPQGPKGEKGANGKRGKMGIPGAAGNPGERGEKGDHGELGLQGNEGPPGQKGEKGDKGDVSNDVLLAGAKGDQGPPGPPGPPGPPGPPGPPGSR

(19) United States
(12) Patent Application Publication
(10) Pub. No.: US 2010/0129288 A1
(43) Pub. Date: May 27, 2010

(54) Gliomedin, Fragments Thereof and Methods of Using Same

(76) Inventors: Elior Peles, Rehovot (IL); Yael Eshed, Moshav Orot (IL)

Correspondence Address: ROTHWELL, FIGG, ERNST & MANBECK, P.C., 1425 K STREET, N.W., SUITE 800, WASHINGTON, DC, 20005, US

(21) Appl. No.: 11/993,711

(22) PCT Filed: Jun. 28, 2006
(86) PCT No.: PCT/IL2006/000753

§ 371 Date: Feb. 2, 2010

Related U.S. Application Data
(60) Provisional application No. 60/694,438, filed on Jun. 28, 2005.
(60) Provisional application No. 60/696,782, filed on Jul. 7, 2005.

(51) Int. Cl. A61K 51/10; C07K 14/47; C07H 21/04; A61K 38/17; C12N 15/63; A61K 31/7088; C12N 5/10; C07K 16/00; C07H 21/02; A61K 35/12; A61K 49/00; C12N 5/071; A61P 25/00

(52) U.S. Cl. 424/1.49; 530/356; 536/23.1; 514/12; 435/320.1; 514/44 R; 435/325; 530/388.1; 530/387.3; 530/391.3; 536/24.5; 424/93.21; 424/9.1; 435/375

(57) ABSTRACT

The present invention relates to pharmaceutical compositions comprising gliomedin and active fragments thereof, polynucleotides encoding gliomedin, host cells expressing gliomedin, antibodies to gliomedin and small interfering RNA (siRNA) molecules that down regulate the expression of gliomedin and methods of using same. The present invention also provides methods of treating and diagnosing neurological disorders using the pharmaceutical compositions and antibodies of the invention.

### FIELD OF THE INVENTION

The present invention relates to pharmaceutical compositions comprising gliomedin and active fragments thereof, polynucleotides encoding gliomedin, host cells expressing gliomedin, antibodies to gliomedin and small interfering RNA (siRNA) molecules that down regulate the expression of gliomedin and methods of using same. The present invention also provides methods of treating and diagnosing neurological disorders using the pharmaceutical compositions and antibodies of the invention.

### BACKGROUND OF THE INVENTION

Rat gliomedin (Database Accession No. AY266116.1-GI:30351047), discovered by the inventors of the present invention, was initially defined as a putative vertebrate ortholog of UNC-122, a phylogenetically conserved type II transmembrane protein with collagen repeats and a cysteine-rich olfactomedin domain (Loria et al. The Journal of Neuroscience, 2004, 24:2191-2201). Graveel et al. (Oncogene 22: 1730-1736, 2003) identified the amino acid sequence of rat gliomedin (Database Accession No. NP_{—}852047) and named it collomin or CRL-L2 (cancer related gene-liver 2). Graveel et al. found that the expression of collomin is upregulated during the development of hepatocellular carcinoma and concluded that this molecule is an excellent marker for this disease. Graveel et al. (ibid) also identified the amino acid sequence of human collomin (Database Accession No. NP 861454). The above publications fail to disclose or suggest that certain gliomedin fragments are particularly active. Moreover, these publications do not disclose or even suggest that gliomedin is involved in the development and maintenance of the nervous system.

Numerous methods of treatment of human neurologic damage, in particular a method for increasing regeneration and repair of damaged neurons and nerve axon myelin coatings, are proposed in the art. For example, U.S. Pat. No. 6,664,266 discloses a method for promoting regenerative growth of an adult human central nervous system by delivering to the axon a therapeutically effective amount of a specific inhibitor of protein kinase C. U.S. Pat. No. 6,569,423 discloses a method of regenerating nervous tissue by contacting the tissue with Schwann cells that express ΔSCIP (also known as Oct-6 and Tst-1). U.S. Pat. No. 6,569,419 discloses a method for promoting the expression of myelin or Protein Zero in Schwann cells using Zcyto7 or IL-17. U.S. Pat. No. 6,512,004 discloses a method for promoting growth of a mammalian central nervous system neural cell subject to growth inhibition by an endogenous neural cell growth repulsion factor, the method comprising contacting the cell with an effective amount of an activator of a cyclic nucleotide dependent protein kinase, wherein the activator comprises an active component selected from a cyclic nucleotide analog and an activator of a cyclic nucleotide cyclase.

U.S. Pat. No. 6,576,607 discloses a method for promoting neural growth in vivo in the mammalian central nervous system by administering a neural cell adhesion molecule, such as NrCAM, to overcome inhibitory molecular cues found on glial cells and myelin and thus to promote neural growth.

Nowhere in the background art is it taught or suggested that gliomedin can induce ion-channel organization along the axons, elicit formation of myelin, initiate node formation in the nervous system and thus can be used for organization of the central and peripheral nervous system.

The aforementioned methods do not meet the requirement for regulating the formation of ion-channels that are essential molecules in the development of myelinated nerves at all developmental stages.

A paper by the inventors of the present invention published after the priority date of the present application describes the ability of gliomedin to induce ion-channel organization along the axons, to elicit formation of myelin and to initiate node formation in the nervous system (Eshed et al., Neuron 47:215-229, Jul. 21, 2005).

### SUMMARY OF THE INVENTION

The present invention provides isolated polypeptide comprising active gliomedin fragments, particularly, but not limited to, the extracellular domain of gliomedin and fragments thereof. The invention further provides polynucleotides encoding the polypeptides of the invention, expression vectors comprising said polynucleotides and host cells transfected with said polynucleotides, including but not limited to cells of the nervous system. The invention also provides antibodies to gliomedin and methods of using same for diagnosing neurological damage. The invention further provides methods of using gliomedin or active fragments thereof for treating, alleviating or preventing neurological damage. In addition, the invention provides small interfering RNA (siRNA) molecules that down regulate the expression of gliomedin and methods of treatment of a subject suffering from a condition associated with elevated levels of gliomedin using the siRNA molecules of the invention.

The present invention is based in part on the unexpected discovery that gliomedin, or fragments thereof, are required for initiating node formation. Furthermore, suppressing the expression of gliomedin was found to cause the abolishment of Na⁺ channels.

The term “node(s)” as used herein refers to the “node(s) of Ranvier” which are short, regularly spaced interruptions (approximately 1 micrometer wide) formed in the myelin sheath along axons. Saltatory conduction along myelinated axons depends on the accumulation of voltage-gated Na⁺ channels at these nodes.

The present invention also shows for the first time that gliomedin is a glial ligand for neurofascin and NrCAM, two axonal IgCAMs that are present at the nodes of Ranvier and are considered as the initial site for assembly of Na⁺ channel clusters and as having an influence on neural growth at the central nervous system (CNS). Without wishing to be bound by any theory or mechanism, the ability of gliomedin to bind neurofascin and NrCAM may be the key to its pivotal role in the regulation and the development of the nervous system as disclosed in the present invention.

The unexpected findings of the present invention indicate that gliomedin is involved in the development and maintenance of the nervous system. These findings include the ability of gliomedin to mediate assembly of the nodes of Ranvier in the peripheral nervous system (PNS). Gliomedin also mediates axon-glia interaction during development. In addition gliomedin participates in maintaining the position of the microvilli towards the nodal axolemma in the mature nerve. Gliomedin further induces formation of Na⁺ channels and moreover regulates clustering of Na⁺ channels.

According to one aspect, the present invention provides an isolated polypeptide comprising the extracellular domain of human gliomedin, a fragment, an analog or a derivative thereof.

According to one embodiment, the isolated polypeptide comprises the entire extracellular domain as set forth in SEQ ID NO:18, a fragment, an analog or a derivative thereof. According to another embodiment, the isolated polypeptide comprises the OLF domain as set forth in SEQ ID NO:19, a fragment, an analog or a derivative thereof. According to yet another embodiment, the isolated polypeptide comprises the collagen repeat domain as set forth in SEQ ID NO:20, a fragment, an analog or a derivative thereof.

According to yet another embodiment, the isolated polypeptide comprises a chemical modification selected from the group consisting of: glycosylation, oxidation, permanent phosphorylation, reduction, myristylation, sulfation, acylation, acetylation, ADP-ribosylation, amidation, hydroxylation, iodination, methylation, and derivatization by blocking groups.

According to alternative embodiments, the isolated polypeptide is either isolated from native cells, or produced by recombinant or synthetic methods.

According to another aspect, the present invention provides an isolated polynucleotide encoding a polypeptide comprising the extracellular domain of human gliomedin, a fragment, an analog or a derivative thereof, wherein said polypeptide comprises the amino acid sequence selected from the group consisting of: SEQ ID NO:18, SEQ ID NO:19 and SEQ ID NO:20.

According to another aspect, the present invention provides an expression vector comprising the isolated polynucleotides of the invention. According to one embodiment, the expression vector further comprises at least one regulatory element operatively linked thereto, the at least one regulatory element is selected from the group consisting of: promoters, enhancers, selection genes, reporter genes, a signal peptide, a recombinase gene and polyA transcription terminator.

According to yet another aspect, the present invention provides a pharmaceutical composition comprising as an active ingredient the expression vector of the invention.

According to yet another aspect the present invention provides a host cell expressing gliomedin or an active fragment thereof. According to one embodiment, the host cell is transfected with the expression vector of the invention. According to another embodiment, the host cell is selected from the group consisting of: a prokaryotic cell, a eukaryotic cell, a somatic cell, a germ cell, a neuronal cell, a pluripotent stem cell and a nerve progenitor cell. According to yet another embodiment, the neural cell is selected from the group consisting of: Schwann cell, myelinating Schwann cell, glial cell and dorsal root ganglion neuron.

According to another aspect, the present invention provides a pharmaceutical composition comprising as an active ingredient the isolated polypeptide of the invention and a pharmaceutically acceptable carrier.

It is to be understood explicitly that the scope of the present invention encompasses homologs, analogs, variants and derivatives, including shorter and longer polypeptides, proteins and polynucleotides, as well as polypeptide, protein and polynucleotide analogs with one or more amino acid or nucleic acid substitution, as well as amino acid or nucleic acid derivatives, non-natural amino or nucleic acids and synthetic amino or nucleic acids as are known in the art, with the stipulation that these variants and modifications must preserve the therapeutic capacities of gliomedin in the treatment of neurological damage. Specifically, any active fragments of the gliomedin as well as extensions, conjugates and mixtures are disclosed according to the principles of the present invention. Particularly, any active fragment derived from the extracellular domain of gliomedin is included within the scope of the invention, including, but not limited to, the entire extracellular fragment as set forth in SEQ ID NO:18, the OLF domain as set forth in SEQ ID NO:19 or the collagen repeat domain as set forth in SEQ ID NO:20.

According to yet another embodiment, the pharmaceutical composition further comprises a pharmaceutically acceptable carrier. According to yet another embodiment, the pharmaceutically acceptable carrier is selected from the group consisting of diluent, solubilizer, emulsifier, excipient, preservative, adjuvant and thickener.

According to yet another aspect the present invention provides a monoclonal anti-gliomedin antibody capable of binding gliomedin or an extracellular fragment thereof. According to one embodiment, the antibody being capable of binding an extracellular fragment comprising the amino acid sequence set forth by SEQ ID NO:18. According to another embodiment the antibody is selected from the group consisting of: humanized antibody, full-length antibody or an antibody fragment. According to yet another embodiment, the antibody fragment is selected from the group consisting of: single chain antibody, Fab′, F(ab′)₂ and Fᵥ.

According to yet another embodiment, the antibody specifically binds to an antigenic determinant of gliomedin. According to yet another embodiment, the antibody is labeled with a detectable label selected from the group consisting of: ultrasound contrast agents, radionuclides, dyes, contrast agents for magnetic resonance imaging, fluorescent compounds and paramagnetic metals. According to yet another embodiment the antibody is a humanized antibody.

According to yet another embodiment, the antibody being identical in function or activity to the antibody produced by cells deposited with the ATCC, deposition #______,

According to a further aspect, the present invention provides a double stranded siRNA molecule that down regulates expression of gliomedin, wherein:
(a) each strand of said siRNA molecule is independently about 15 to about 30 nucleotides in length; and
(b) one strand of said siRNA molecule comprises a nucleotide sequence having sufficient complementarity to an RNA of gliomedin or a fragments thereof.

According to one embodiment, the siRNA molecule comprises at least one modification selected from the group consisting of: 2′-sugar modification, at least one nucleic acid base modification, and at least one phosphate backbone modification.

According to another embodiment, the siRNA molecule comprises an oligonucleotide selected from SEQ ID NO:3, SEQ ID NO:4 and SEQ ID NO:5.

According to yet another aspect, the present invention provides a method of down-regulating gliomedin expression in a cell, comprising contacting the cell with the siRNA molecules of the invention, under conditions suitable for down-regulating of gliomedin expression.

According to yet another aspect the present invention provides a method of treating neurological damage in a subject in need thereof, the methods comprising administering to the subject the pharmaceutical compositions of the invention.

According to one embodiment, the neurological damage is selected from the group consisting of: multiple sclerosis, axonal injury, lack of ion channel in axolemma, disordered axonal clustering of Na⁺ channels, damaged initial Schwann cell myelination, damaged initial nerve development and demyelination-associated disease.

According to yet another embodiment, the demyelination-associated disease is selected from the group consisting of: diabetic neuropathy, Guillain-Barre Syndrome, chronic demyelinating disease, acute demyelinating polyneuropathy, human immunodeficiency viral demyelinating neuropathy, demyelination caused by trauma, inherited neuropathies referred to as Charcot-Marie-Thooth disease (CMT), hereditary motor syndrome and sensory neuropathy (HMSN).

According to another embodiment, administering is carried out by a method selected from the group consisting of: oral administration, intravenous injection, intramuscular injection and intrathecal injection.

According to yet another aspect the present invention provides a method of treating neurological damage in a subject in need thereof, the methods comprising transplanting the host cells of the invention into the subject.

According to one embodiment, the transplanted host cells are autologous cells genetically modified to express gliomedin or an active fragment thereof. According to another embodiment, the host cells are transplanted at or near at least one predetermined locus. According to yet another embodiment, the at least one predetermined locus lacks sufficient levels of Na⁺ channels.

According to yet another aspect, the present invention provides a method of diagnosing neurological damage, the method comprising:
(a) administering to a subject in need thereof an anti-gliomedin antibody of the invention;
(b) detecting the amount of said antibody by imaging techniques; and, optionally,
(c) evaluating the localization and/or amount of bound antibody and comparing said localization and/or amount with the localization and/or amount of bound antibody in a control healthy subject.

According to one embodiment, the method further comprises administering to said subject a clearing agent and allowing said clearing agent to clear non-localized antibody. According to another embodiment, the clearing agent is an anti-idiotypic antibody or antigen-binding antibody fragment.

According to yet another embodiment, the anti-gliomedin antibody is conjugated to a diagnostic agent. According to yet another embodiment, the diagnostic agent is selected from the group consisting of radionuclides, dyes, contrast agents, fluorescent compounds or molecules and enhancing agents useful for magnetic resonance imaging (MRI).

According to yet another embodiment, the diagnostic agent is a radionuclide useful in positron emission, said radionuclide selected from the group consisting of F-18, Mn-51, Mn-52m, Fe-52, Co-55, Cu-62, Cu-64, Ga-68, As-72, Br-75, Br-76, Rb-82m, Sr-83, Y-86, Zr-89, Tc-94m, In-110, I-120 and I-124.

According to yet another embodiment, said diagnostic agent is useful in magnetic resonance imaging techniques, said agent comprises metals selected from the group consisting of gadolinium, manganese, iron, chromium, copper, cobalt, nickel, dysprosium, rhenium, europium, terbium, holmium and neodymium.

According to yet another embodiment, said diagnostic agent is a radionuclide useful in gamma-ray detection and wherein said radionuclide is selected from the group consisting of Cr-51, Co-57, Co-58, Fe-59, Cu-67, Ga-67, Se-75, Ru-97, Tc-99m, In-111, In-114m, 1-123, I-125, I-131, Yb-169, Hg-197 and T1-201.

According to yet another embodiment, the diagnostic agent is administered by a method selected from the group consisting of: intravenous bolus, intravenous perfusion, intraarterial, intrapleural, intraperitoneal, intrathecal and subcutaneous.

According to yet another embodiment, the method is used in conjunction with intraoperative probes, endoscopic and laparoscopic uses, and in methods for imaging normal organs.

Other objects, features and advantages of the present invention will become clear from the following description and drawings.

### BRIEF DESCRIPTION OF THE DRAWINGS

FIG. 1 presents binding of NF186-Fc and NF155-Fc to DRG explants (A-D, scattered dots), binding of NF155-Fc to a Schwann cell that is aligned with a neurofilament-labeled axon, including an enlarged image of the cell process spiraling around the axon is shown in the inset (E) and binding of NF155-Fc (F), NF186-Fc (G), or L1-Fc (H) to isolated rat Schwann cells (F-H). Cells were labeled with antibodies to neurofilament to detect neurons (A, C) or S100 to detect Schwann cells (B, D). Cell nuclei are labeled with DAPI (A-H, large oval/round objects). Scale bars: A-D, 50 μm; E, 20 μm; F-H, 50 μm.

FIG. 2A presents binding of NF-Fc to COS-7 cells transfected with one of the primary Schwann cDNA pools (1st), or a single cDNA isolated after three additional rounds of screening (3rd).

FIG. 2B presents the amino acid sequence of rat gliomedin (NP 852047), particularly the transmembrane domain (boxed amino acids 26-38; SEQ ID NO:7), collagen repeat (upperlined amino acids; SEQ ID NO:8), the olfactomedin domain (boxed amino acids 298-543; SEQ ID NO:9) and the entire extracellular domain (amino acids 39-549; SEQ ID NO:10).

FIG. 2C illustrates the domain organization of gliomedin (GLDN) compare to several representatives of the olfactomedin family. Olfactomedin domain is shown as an ellipse and the collagen-like repeat as a square. LPHN-latrophilin; OLFM-olfactomedin; CG6867 encodes a putative transmembrane protein in Drosophila melangoster. The gliomedin (GLDN) subgroup also contains UNC122 and COF-1 in C. elegans.

FIG. 2D exhibits binding of neurofascin-Fc to gliomedin-expressing Schwann cells. Isolated rat Schwann cells were allowed to bind NF155-Fc (upper panel) and then fixed and stained using an antibody to gliomedin (lower panel) and Dapi to label nuclei (large oval objects).

FIG. 2E exhibits binding of different Fc fusion proteins containing the extracellular domains of the NF155, NF186, L1, NrCAM or contactin (Con-Fc) to COS7 cells expressing gliomedin (upper row) and expression of gliomedin on the cell surface was detected using specific antibodies (lower row).

FIG. 2F is a schematic presentation of the soluble gliomedin Fc-fusion: ECD—extracellular domain, COL—collagen repeat, OLF—olfactomedin domain. The Fc region is depicted as a box.

FIGS. 2G-H show binding of ECD-Fc, OLF-FC, or COL-Fc to COS7 cells expressing neurofascin (G, upper row) and expression of neurofascin in the transfected cells was determined using specific antibodies (G, lower panel) and binding of ECD-Fc (upper panel) and COL-Fc (Lower panel) to sections of rat sciatic nerve. The location of the paranodal junctions is labeled with antibodies to Caspr. Scale bars: D, E, G: 10 μm; H: 5 μm.

FIGS. 3A-3D show gliomedin localization at the nodes of Ranvier in the PNS applying double immunofluorescence labeling of teased adult rat sciatic nerve with an antibody to gliomedin (arrows, gliomedin or GLDN in all panels), and antibodies to neurofilament (A), MAG (B), Caspr (C) or pan neurofascin (D) as indicated (arrow heads). The pan-neurofascin antibody recognizes both neurofascin isoforms, NF186 and NF155 that are found at the nodes and paranodal junction, respectively. Scale bars: 10 μm

FIG. 3E presents consecutive optical sections through the nodes and the paranodes of teased sciatic nerves labeled for gliomedin (GLDN) and pan-neurofascin (NP), along with a schematic drawing showing the location of the section.

FIGS. 3F-3J are higher magnification of the nodal region in teased fiber preparations (F-I) or cross section (J) of sciatic nerves, double-labeled with an antibody to gliomedin (arrows) and Na⁺ channels (F; arrow heads and J), Ankyrin G (G; arrow heads), NrCAM (H; arrow heads) or NF186 (I). Insets in panels F-H and J, show merged images in which the channels were shifted. The dotted line in panel I describes the occasional labeling, by anti-gliomedin antibodies, at the nodes outside the nodal axolemma. Scale bars: F-H—5 μm.

FIGS. 3K-M present a horizontal section of the ventral region of the spinal cord, including the ventral roots was immunolabeled with an antibody to gliomedin (arrows) and Caspr. The border between the CNS and the PNS is depicted with a dashed line. L-M are higher magnification of the areas marked in panel K.

FIG. 4 exhibits immunofluorescence labeling of teased sciatic nerve (A-B) or cross section (C), using an antibody to gliomedin (in green) and antibodies to moesin (A), ezrin (B), or claudin-2 (C).

FIGS. 4D-4H show immunoelectron microscopy images of gliomedin in adult sciatic nerve. Immunogold labeling of gliomedin on cross (D-E), or longitudinal (F-H) sections through the nodes. Panels F and H show higher magnifications of the squares labeled in G. Scale bars: D, 0.5 μm; E, F, H, 0.2 μm; G, 1 μm.

FIG. 5A presents immuno-fluorescence labeling of longitudinal sections of 1, 4, 8, 10 and 14 day-old rat sciatic nerve with antibodies to gliomedin and Na⁺ channels (upper panel) or to Caspr (lower panel). Inset depicts another field of view. Scale bar: 20 μm.

FIG. 5B are images of developing nodes from longitudinal sections of P4 nerve double-labeled with antibodies to gliomedin (left column) and Na⁺ channels (middle column). Merged images are shown on the right panel of each row. Different stages, from the immature binary form (top) to the mature focal node (bottom) are depicted. Scale bar: 5 μm.

FIG. 5C presents the localization of gliomedin and Na⁺ channels during development: sciatic nerve sections labeled for gliomedin and Na⁺ channels and the number of clusters containing gliomedin (Gldn), Na⁺ channels (NaCh) or both (Gldn/NaCh) were counted. The results are shown as a percentage of the total count (n=200 sites).

FIG. 5D presents developmental clustering of gliomedin compared to Caspr: Sciatic nerve sections were labeled for gliomedin and Caspr and the numbers of sites that contained each protein alone (Caspr or Gldn), or sites containing gliomedin flanked by a single or double Caspr staining were scored (n=200 sites).

FIG. 5E presents immunolabeling of myelinating dorsal root ganglion explants 5 days after the induction of myelination with antibodies to gliomedin (arrows), MAG (low panel) and MBP (upper panel). The edges of the Schwann cells are marked with arrowheads. Scale bar: 20 μm.

FIG. 6 exhibits redistribution of gliomedin and inhibition of node formation upon treatment of myelinating cultures with NF-Fc: A—immunofluorescence staining of myelinating DRG cultures that were grown in the presence of NF-Fc or a control Fc for 12 days, using antibodies to gliomedin (GLDN), Na⁺ channels (NaCh) and MBP. Higher magnifications of the nodal areas (marked with arrowheads) are shown at both sides of each panel. Arrows in the third panel depict small clusters of gliomedin that are found along the internodes; Scale bars: 20 μm. B upper panel—a myelinating Schwann cell from NF-Fc treated culture immunolabeled with antibodies to MBP (arrow heads) and gliomedin (arrows). N indicates the location of the nucleus; B lower panel—inverted image of another myelinating cell stained for gliomedin showing clusters of gliomedin on the outer (abaxonal) region of the myelin unit along the entire internodes. Scale bar: 5 μm. C—the number of nodal sites (mean±SD) labeled for βIV spectrin and gliomedin in control (Fc), or NF-Fc treated cultures is shown as a percentage of the total sites counted (n=4; t-test p<0.006); D-F show control (Fc) or NF-Fc treated cultures were immunolabeled with antibodies to MBP, Caspr, Gliomedin (GLDN), βIV-spectrin (Spec), ankyrin G (AnkG), or phosphorylated ERM (PERM). D—lower panel only MBP is detected while in the upper (Fe) panel all three signals are observed; E—left (N) section, upper panel only Spec is observed, no staining is observed in the low (NF-Fc) panel; middle (PN) section, only Caspr staining is observed in upper and lower panels; F—in left section upper panel both GLDN and PERM staining are observed, left section lower panel, only PERM staining is detected, right section of upper and lower panels presents only MBP staining. The position of the nodes (N), paranodes (PN) and internodes (IN) is marked with vertical lines.

FIG. 7 exhibits DRG-derived Schwann cells infected with retroviruses containing a GFP and siRNA designed to inhibit the expression of gliomedin (A, RNAi#1 (SEQ ID NO: 3); B,D,F RNAi#3 (SEQ ID NO: 4) or myelin associated glycoprotein (MAG; C,E,G). Cells were allowed to myelinate for 12 days and then fixed and immunolabeled with antibodies to MBP (staining the internodes), gliomedin (GLDN) and Na⁺ channels (NaCh) or neurofascin (NF). The infected cells were identified by monitoring the expression of GFP (large oval objects). The right half of the internodes of the infected cell in panels B and C are shown at a higher magnification in D and E. Arrowheads mark the location of the nodes at both sides of MBP-labeled internodes. Arrows in panels A, B, D and F indicate the clustering of gliomedin and Na⁺ channels or neurofascin at the edge of non-infected myelinating Schwann cells. Insets depict higher magnification of the indicated nodes; insets in A, D and E show images after shifting the red and blue channels to show the labeling of the individual components, whereas the insets in F and G show the merge images. Scale bars: 5 μm.

FIG. 8A shows binding of Fc-fusion proteins containing the olfactomedin domain of gliomedin (OLF-Fc) or the extracellular domain of IgSF4 (IgSF4-Fc; control) to DRG axons (upper panels), along with immunofluorescence labeling for neurofascin (A; middle panels) and the merge images (lower panels).

FIG. 8B shows the merged images for each antibody in OLF-Fc treated DRG neurons fixed and immunolabeled for the bound OLF-Fc (insets Neurofascin, AnkG, Spectrin, Casper—upper raw and insets NaV 1.2 and 4.1B lower raw) Scale bars: 5 μm.

FIG. 8C shows the percentage of the total Fc clusters counted of Na⁺ channels, neurofascin, ankyrin G or βIV spectrin clusters that were found adjacent to clusters of OLF-Fc or IgSF4-Fc. All values are mean±SD (n=3).

FIG. 8D shows gliomedin-induced clusters contained several nodal proteins: upper bands correspond to OLF-Fc; middle bands correspond to NF, NF, NaCh and AnkG, respectively; and lower band correspond to Spectrin or NaCh, as indicated in each panel. Scale bar: 5 μm.

FIG. 9 presents expression analysis of gliomedin mRNA: A. Northern blots containing mRNA from various human tissues were hybridized with a gliomedin-specific probe; B. expression of Gliomedin mRNA in the rat sciatic nerve and brain; C. in situ hybridization analysis of Gliomedin in cross sections of rat DRGs connected to peripheral nerves. Arrowheads mark the location of the sciatic nerve.

FIG. 10 exhibits the specificity of anti-gliomedin antibodies: A. shows teased rat sciatic nerves that were immunolabeled with antibodies to gliomedin (arrows) and Caspr (arrow heads) in the absence (upper panel) or presence (lower panel) of 10 μg/ml of the peptide antigen used to generate the anti-gliomedin antibody. B. is a Western blot analysis of cultured rat Schwann cells using the #720 antibody to gliomedin in the absence (−) or presence (+) of the immunizing peptide. The location of molecular weight marker is shown on the right in kDa.

FIG. 11 presents the amino acid sequence of human gliomedin (SEQ ID NO:15), including the transmembrane domain (boxed amino acids 16-38), collagen repeat (upperlined amino acids; SEQ ID NO:20), the olfactomedin domain (boxed amino acids 302-546; SEQ ID NO:19) and the entire extracellular domain (amino acids 39-551; SEQ ID NO:18).

FIG. 12 presents the amino acid sequence alignment of rat gliomedin (SEQ ID NO:1) and human gliomedin (SEQ ID NO:14).

### DETAILED DESCRIPTION OF THE INVENTION

### Definitions

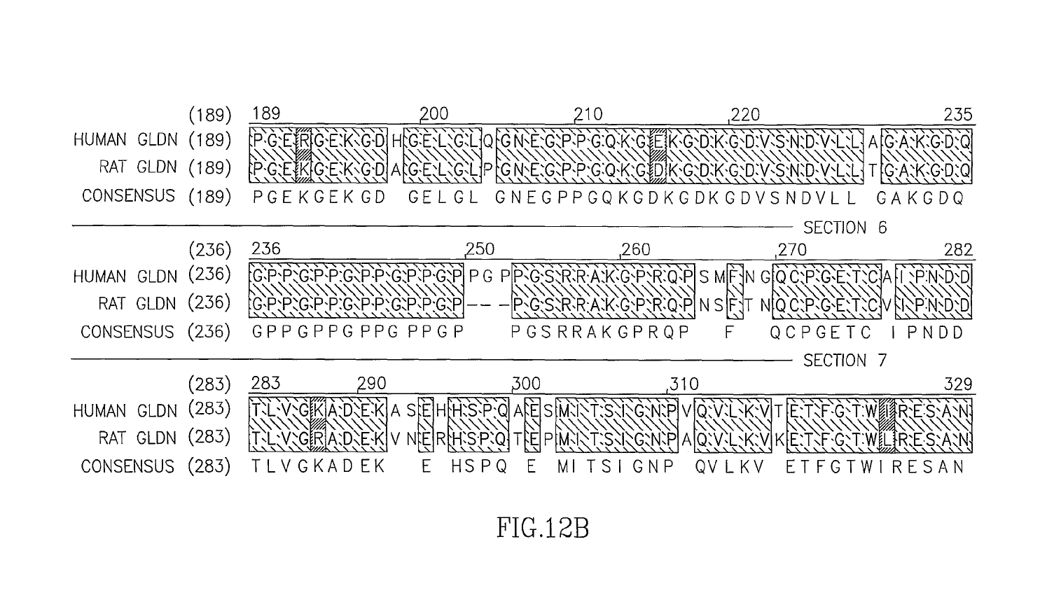

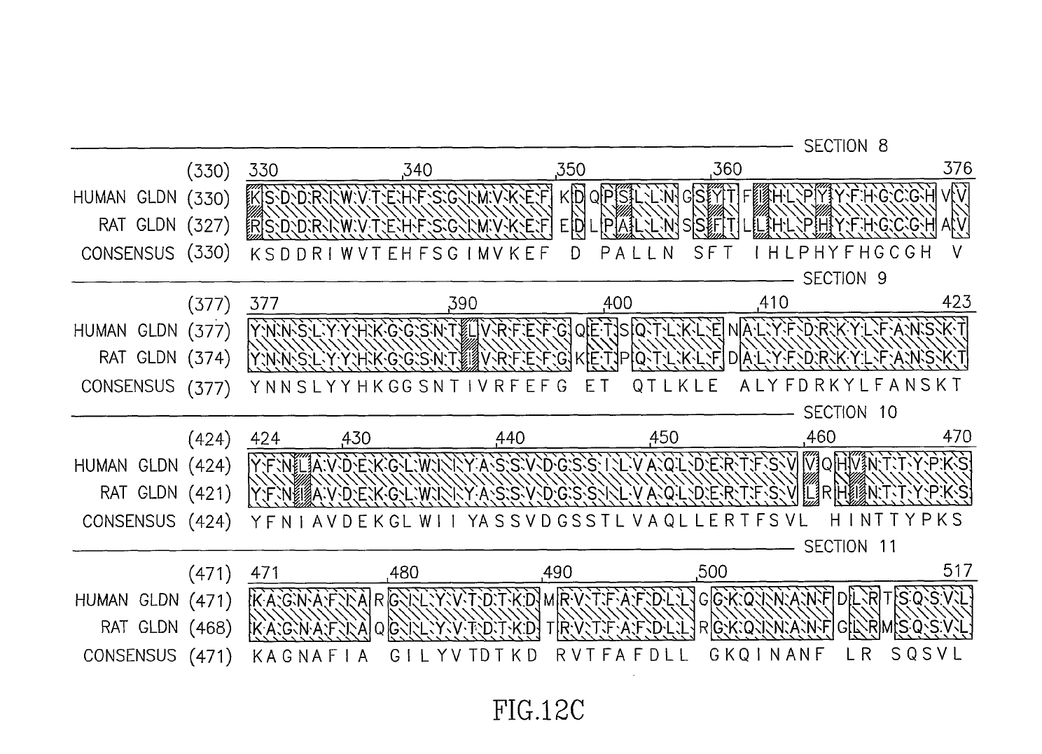

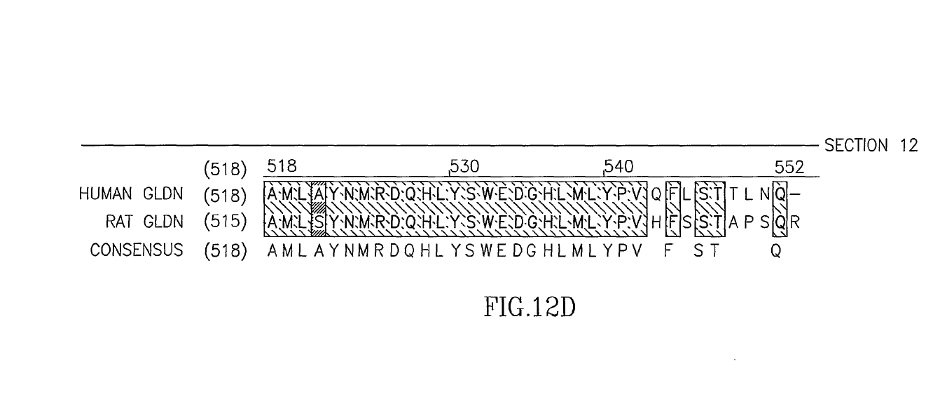

The term “gliomedin” as used herein refers to gliomedin protein or functional fragments thereof, including, but not limited to, the soluble extracellular domain of gliomedin and the olfactomedin domain of gliomedin, also termed hereinafter OLF-Fc. The amino acid sequence of gliomedin protein and functional fragments thereof include the amino acid sequences set forth in SEQ ID NO: 1, SEQ ID NO: 8, SEQ ID NO: 9, SEQ ID NO: 10, SEQ ID NO: 14, SEQ ID NO: 18, SEQ ID NO: 19 and SEQ ID NO: 20. However the term “gliomedin” extends to homologs, analogs, variants and derivatives, including shorter and longer polypeptides and proteins with one or more amino acid substitution, non-natural amino acid(s) with the stipulation that these variants and modifications must preserve the capacities of gliomedin, e.g. node formation. Particularly, the terms “analog” and “variant” are interchangeably used herein to describe altered gliomedin or fragments thereof, including an amino acid sequence selected from the group consisting of: SEQ ID NO: 14, SEQ ID NO:18, SEQ ID NO:19 and SEQ ID NO: 20 altered by amino acid substitutions, additions, deletions, or chemical modifications. By using “amino acid substitutions”, it is meant that functionally equivalent amino acid residues are substituted for residues within the sequence resulting in a silent change. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such substitutions are known as conservative substitutions. Additionally, a non-conservative substitution may be made in an amino acid that does not contribute to the biological activity, e.g., node formation induced by gliomedin or a fragment thereof. It will be appreciated that the present invention encompasses rat gliomedin including the amino acid as set forth by SEQ ID NO: 1, SEQ ID NO: 8, SEQ ID NO: 9 and SEQ ID NO: 10. An amino acid sequence alignment of rat and human gliomedin is illustrated in FIG. 12.

The terms “neurological dysfunction” or “neurological disorder” are interchangeably used herein to describe any disease or disorder associated with loss of neural organization due to absence or lack of sufficient amount of sodium channels.

The terms “sufficient”, “effective amount” or “sufficient amount” are interchangeably used herein to describe the amount required to overcome a physiological disorder.

As defined herein, “nervous tissue” comprises any or all of the following: neuronal cells, Schwann cells, stellate cells, neuroglial cells, granule cells, ganglia cells, grey matter, white matter, myelin, neurilemma, axons, dendrites, motor neurons, fibrils and fibular processes.

As used herein, “neural cells” are any of the cells that constitute nervous tissue and the tissue that supports it including, but not limited to, Schwann cells, stellate cells; neuroglial cells, granule cells and ganglia cells. The granule cells may be cerebellar granule cells or cerebral granule cells.

Inducing “nervous tissue regeneration” is herein defined as inducing one or more of: the myelination of a nerve, the growth of neurons, the growth of the axons or dendrites of the nerve, the growth of fibrils of neuroglia, the growth of stellate cells, the growth of fibular processes of neuroglia, the remyelination of grey matter, and the remyelination of white matter. The neuronal regeneration may take place in nerves of both the central nervous system and the peripheral nervous system.

As used herein, “growth” may be defined as an increase in thickness, diameter, and length of the nerve fibers or the myelin or neurilemma coverings, and the supporting fibrils and fibular processes. The definition of “growth” as used herein also includes an increase in the numbers of Schwann cells, stellate cells or neuroglial cells present on or supporting a nerve.

### Preferred Modes for Carrying Out the Invention

Saltatory conduction along myelinated axons depends on the accumulation of voltage-gated Na⁺ channels at short, regularly spaced interruptions in the myelin sheath known as the nodes of Ranvier. The saltatory conduction is the electrical impulse that jumps from one node to the next at a rate as fast as 120 meters/second. At the nodes, these channels are found in a multiprotein complex that includes cell adhesion molecules and cytoplasmic adaptor proteins. (Poliak and Peles, 2003, Nat Rev Neurosci 4, 968-980) Na⁺ channels are associated with NrCAM and the 186-kDa isoform of neurofascin (NF186), two IgCAMs that are concentrated at the nodal axolemma—the plasma membrane of an axon (also called Mauthner's sheath). The interaction between these IgCAMs and Na channels occurs directly or through ankyrin G, an adaptor that links integral membrane proteins to the cortical cytoskeleton and is enriched at the nodes. Ankyrin G binds βIV spectrin, further anchoring the nodal Na⁺ channel and IgCAMs to the axonal cytoskeleton.

The position of the nodes is tightly regulated by the overlying glial cells and is not intrinsically specified by the axon (Poliak and Peles, 2003; ibid). During the development of peripheral myelinated nerves, Na⁺ channels cluster at sites adjacent to the edges of processes extended by myelinating Schwann cells, indicating that these Na⁺ clusters are positioned by a direct glial cell contact. This conclusion is also supported by freeze-fracture analysis, demonstrating that the nodal specialization is always associated with Schwann cell processes. In agreement, Na⁺ channels clusters are absent after ablation of Schwann cells; they are dispersed during acute demyelination, and reappear during remyelination. In contrast to the defined requirement for direct axon-glia contact for node formation in the PNS, Na⁺ clustering in the CNS might be induced by soluble factors secreted by oligodendrocytes. Whereas CNS nodes are contacted by processes of perinodal astrocytes and NG2-positive cells, PNS nodes are abutted by Schwann cell microvilli that emanate from the outer collar of the cell. These microvilli appear relatively late during the maturation of the myelin unit and develop from an early glial process that contact the nodal. Schwann cell microvilli contain ERM proteins (ezrin, radixin and moesin), the ezrin binding protein EBP50 and Rho-A GTPase, as well as syndecans and dystroglycan. ERM-positive processes have been shown to align with the developing nodes and thus, were suggested to mediate axon-glia interactions necessary for the clustering of Na⁺ channels. Disruption of Schwann cell microvilli resulted in a striking reduction in nodal Na⁺ channel clustering.

Previous studies suggested that during the development of peripheral nodes, Na⁺ channels are recruited to clusters of IgCAMs and ankyrin G that were first positioned by glial processes. Addition of a soluble NrCAM, or neurofascin to myelinating dorsal root ganglia cultures inhibits Na⁺ clustering. In vivo, the appearance of Na⁺ channels and ankyrin G at the nodes is delayed in NrCAM null mice, further indicating a role for the nodal IgCAMs in node formation. The present invention discloses that gliomedin is a Schwann cell ligand for both neurofascin and NrCAM and is localized to the nodal microvilli from the onset of myelination. The present invention indicates that Schwann cell-axon interactions mediated by gliomedin trigger the molecular assembly of the nodes of Ranvier in the PNS.

Pharmaceutical Compositions

The present invention provides a pharmaceutical composition comprising a therapeutically effective amount of gliomedin or an active fragment thereof. The protein of the present invention, or a pharmacologically acceptable salt thereof may be mixed with an excipient, carrier, diluent, and optionally, a preservative or the like, pharmacologically acceptable vehicles as known in the art.

As used herein, the term “suitable pharmaceutically acceptable carrier” encompasses any of the standard pharmaceutically accepted carriers, such as phosphate buffered saline solution, water, emulsions such as an oil/water emulsion or a triglyceride emulsion, various types of wetting agents, tablets, coated tablets and capsules. An example of an acceptable triglyceride emulsion useful in intravenous and intraperitoneal administration of the compounds is the triglyceride emulsion commercially known as Intralipid™.

Typically such carriers contain excipients such as starch, milk, sugar, certain types of clay, gelatin, stearic acid, talc, vegetable fats or oils, gums, glycols, or other known excipients. Such carriers may also include flavor and color additives or other ingredients.

The pharmaceutical composition of the invention may further include diluents, preservatives, solubilizers, emulsifiers and/or adjuvants Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the compound, complexation with metal ions, or incorporation of the compound into or onto particulate preparations of polymeric compounds such as polylactic acid, polglycolic acid, hydrogels, etc, or onto liposomes, micro emulsions, micelles, unilamellar or multi lamellar vesicles, erythrocyte ghosts, or spheroplasts. Such compositions will influence the physical state, solubility, stability, rate of in vivo release, and rate of in vivo clearance of the compound or composition. The choice of compositions will depend on the physical and chemical properties of the compound capable of alleviating the symptoms of the neuronal disorder.

The pharmaceutical composition of the invention may be provided in the form of controlled- or sustained release composition. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal and oral.

Portions of the compound of the invention may be “labeled” by association with a detectable marker substance (e.g., radiolabeled with 125I or biotinylated) to provide reagents useful in detection and quantification of gliomedin or its receptor bearing cells or its derivatives in solid tissue and fluid samples such as blood, cerebral spinal fluid or urine.

Examples of excipients include glucose, mannitol, inositol, sucrose, lactose, fructose, starch, cornstarch, microcrystalline cellulose, hydroxypropylcellulose, hydroxypropyl-methylcellulose, polyvinylpyrrolidone and the like. Optionally, a thickener may be added, such as a natural gum, a cellulose derivative, an acrylic or vinyl polymer, or the like. The pharmaceutical composition including the peptide may further comprise a biodegradable polymer selected from poly-1,4-butylene succinate, poly-2,3-butylene succinate, poly-1,4-butylene fumarate and poly-2,3-butylene succinate, incorporating the peptide of the invention as the pamoate, tannate, stearate or palmitate thereof. Such compositions are known in the art as described, for example, in U.S. Pat. No. 5,439,688.

The preparation of pharmaceutical compositions comprising peptides is well known in the art, as disclosed for example in U.S. Pat. Nos. 5,736,519, 5,733,877, 5,418,219, 5,354,900, 5,298,246, 5,164,372, 4,900,549 and 4,457,917. Means for processing the pharmaceutical compositions of the present invention include, without limitations, conventional mixing, dissolving, granulating, grinding, pulverizing, dragee-making, levigating, emulsifying, encapsulating, entrapping or lyophilizing processes.

Expression Vectors

This invention also provides expression vectors, such as, a plasmid and a vector, comprising a polynucleotide selected from the group consisting of: SEQ ID NO:2, SEQ ID NOS: 11-13, SEQ ID NO:15, a polynucleotide capable of encoding SEQ ID NO:1, and polynucleotide capable of encoding gliomedin as set forth in SEQ ID NOS: 8-10 and SEQ ID NO:15 or an active fragment thereof. The vector may be a prokaryotic expression vector, a eukaryotic expression vector, a mammalian expression vector, a yeast expression vector, a baculovirus expression vector or an insect expression vector. Examples of these vectors include PKK233-2, pEUK-C1, pREP4, pBlueBacHisA, pYES2, PSE280 or pEBVHis. Methods for the utilization of these replicable vectors may be found in Sambrook, et al., 1989 or in Kriegler 1990.

A “reporter gene”, as defined herein, is a gene encoding a molecule which by its chemical nature, provides an identifiable signal allowing detection of the circular oligonucleotide. Detection can be either qualitative or quantitative. The present invention contemplates using any commonly used reporter genes as well as reporter molecules including radionuclides, enzymes, biotins, psoralens, fluorophores, chelated heavy metals, and luciferin. The most commonly used reporter molecules are either enzymes, fluorophores, or radionuclides linked to the nucleotides which are used in circular oligonucleotide synthesis. Commonly used enzymes include horseradish peroxidase, alkaline phosphatase, glucose oxidase and α-galactosidase, among others. The substrates to be used with the specific enzymes are generally chosen because a detectably colored product is formed by the enzyme acting upon the substrate. For example, p-nitrophenyl phosphate is suitable for use with alkaline phosphatase conjugates; for horseradish peroxidase, 1.2-phenylenediamine, 5-aminosalicylic acid or toluidine are commonly used. The methods of using such hybridization probes are well known and some examples of such methodology are provided by Sambrook et al, 1989.

### Host Cells

The present invention provides a host cell expressing exogenous gliomedin or fragments thereof. According to one embodiment, the host cell is selected from the group consisting of: a eukaryotic cell, a somatic cell, a germ cell, a neuronal cell, pluripotent stem cell and nerve progenitor cell. According to yet another embodiment, the neural cell is selected from the group consisting of: Schwann cell, myelinating Schwann cell, Schwann cell microvilli, glial cell and dorsal root ganglion neuron.

According to another embodiment the host cell comprises an exogenous polynucleotide sequence encoding SEQ ID NO:1, SEQ ID NOS:8-10 and SEQ ID NO:14. According to yet another embodiment the exogenous polynucleotide sequence comprises. SEQ ID NO:2, SEQ ID NOS:11-13 and SEQ ID NO:15.

The host cell may be a human cell which has been stably transformed by a recombinant nucleic acid molecule encoding gliomedin or an active fragment thereof, such as SEQ IN NO:2. The nucleic acid may be operatively linked to a regulatory element as defined herein including but not limited to a promoter, an enhancer, a recombinase gene, a reporter gene, a transactivator transcription factor gene and a transcription factor gene.

### Antibodies

The present invention provides anti-gliomedin antibody that recognize and bind fragments of gliomedin, provided that the antibodies are first and foremost specific for, as defined above, gliomedin. Antibodies of the invention can be produced using any method well known and routinely practiced in the art.

Anti-gliomedin antibody being identical in function or activity to the antibody produced by cells deposited with the ATCC, deposition #______, under the provisions of the Budapest Treaty, (also termed hereinafter MAb94) are also contemplated. Antibody fragments, including Fab, Fab′, F(ab′)₂, and Fv, are also provided by the invention. The term “specific for” indicates that the variable regions of the antibodies of the invention recognize and bind gliomedin exclusively, but may also interact with other proteins (for example, other antibodies in ELISA techniques) through interactions with sequences outside the variable region of the antibodies, and in particular, in the constant region of the molecule.

Antibodies that recognize and bind fragments of gliomedin are also contemplated, provided that the antibodies are first and foremost specific for, as defined above, gliomedin. Antibodies of the invention can be produced using any method well known and routinely practiced in the art.

Non-human antibodies may be humanized by any methods known in the art. In one method, the non-human complementarity determining regions (CDRs) are inserted into a human antibody or consensus antibody framework sequence. Further changes can then be introduced into the antibody framework to modulate affinity or immunogenicity.

For example, U.S. Pat. No. 5,585,089 of Queen et al. discloses a humanized immunoglobulin and methods of preparing same, wherein the humanized immunoglobulin comprises complementarity determining regions (CDRs) from a donor immunoglobulin and heavy and light chain variable region frameworks from human acceptor immunoglobulin heavy and light chains, wherein said humanized immunoglobulin comprises amino acids from the donor immunoglobulin framework outside the Kabat and Chothia CDRs, wherein the donor amino acids replace corresponding amino acids in the acceptor immunoglobulin heavy or light chain frameworks.

U.S. Pat. No. 5,225,539, of Winter, also discloses an altered antibody or antigen-binding fragment thereof and methods of preparing same, wherein a variable domain of the antibody or antigen-binding fragment has the framework regions of a first immunoglobulin heavy or light chain variable domain and the complementarity determining regions of a second immunoglobulin heavy or light chain variable domain, wherein said second immunoglobulin heavy or light chain variable domain is different from said first immunoglobulin heavy or light chain variable domain in antigen binding specificity, antigen binding affinity, species, class or subclass.

Antibodies of the invention are useful for, for example, therapeutic purposes (by modulating activity of gliomedin), diagnostic purposes to detect or quantitate gliomedin, as well as purification of gliomedin. Antibodies are useful for detecting and/or quantitating gliomedin expression in cells, tissues, organs and lysates and extracts thereof, as well as fluids, including serum, plasma, cerebrospinal fluid, urine, sputum, peritoneal fluid, pleural fluid, or pulmonary lavage. Kits comprising an antibody of the invention for any of the purposes described herein are also comprehended. In general, a kit of the invention also includes a control antigen for which the antibody is immunospecific.

Specific binding proteins can be identified or developed using isolated or recombinant gliomedin products, gliomedin variants, or cells expressing such products. Binding proteins are useful for purifying gliomedin products and detection or quantification of gliomedin products in fluid and tissue samples using known immunological procedures. Binding proteins are also manifestly useful in modulating (i.e., blocking, inhibiting or stimulating) biological activities of gliomedin, especially those activities involved in organization of the neural system.

Anti-idiotype antibodies specifically immunoreactive with an antibody of the invention are also comprehended.

Small Interfering RNA (siRNA)

The present invention provides a double stranded siRNA molecule that down regulates expression of gliomedin via RNA interferences, wherein:
(a) each strand of said siRNA molecule is independently about 15 to about 30 nucleotides in length; and
(b) one strand of said siRNA molecule comprises nucleotide sequence having sufficing complementarity to an RNA of said gliomedin.

According to certain embodiments, the siRNA molecule comprises an oligonucleotide selected from SEQ ID NO:3, SEQ ID NO:4 and SEQ ID NO:5. The siRNA molecule of the invention may comprise at least one modification selected from the group consisting of: 2′-sugar modification, at least one nucleic acid base modification, and at least one phosphate backbone modification.

The siRNA molecules of the present invention may be modified to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163).

An RNA of gliomedin refers to an RNA being translated to gliomedin or a fragment of gliomedin, including but not limited to, the gliomedin fragments set forth in SEQ ID NO:1, 8-10, 14 and 18-20.

By “triplex forming oligonucleotides” or “triplex oligonucleotide” is meant an oligonucleotide that can bind to a DNA in a sequence-specific manner to form a triple-strand helix. Formation of such triple helix structure has been shown to inhibit transcription of the targeted gene (e.g. U.S. Pat. No. 7,022,828).

By “double stranded RNA” or “dsRNA” is meant a double stranded RNA that matches a predetermined gene sequence that is capable of activating cellular enzymes that degrade the corresponding messenger RNA transcripts of the gene. These dsRNAs are referred to as short intervening RNA (siRNA) and can be used to inhibit gene expression (see for example International PCT Publication Nos. WO 00/44895; WO 01/36646; WO 99/32619; WO 00/01846; WO 01/29058; WO 99/07409; and WO 00/44914).

“A double stranded RNA that matches a nucleic acid molecule” or “an oligonucleotide that can bind to a DNA in a sequence-specific manner” is meant to describe a double stranded RNA (dsRNA) which have complementarity in a substrate binding region to a specified gene target, and also induce an enzymatic activity which is active to specifically cleave target RNA. That is, the dsRNA molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50% to 75% can also be sufficient complementarity in the present invention (see for example Werner and Uhlenbeck, 1995, Nucleic Acids Research, 23, 2092 2096; Hammann et al., 1999, Antisense and Nucleic Acid Drug Dev., 9, 25 31). The nucleic acids can be modified at the base, sugar, and/or phosphate groups.

By “inhibit” or “down-regulate” it is meant that the expression of the gene, or level of RNAs or equivalent RNAs encoding one or more protein subunits, or activity of one or more protein subunits, such as gliomedin or gliomedin subunit(s), is reduced below that observed in the absence of the nucleic acid molecules of the invention. In one embodiment, inhibition or down-regulation with enzymatic nucleic acid molecule preferably is below that level observed in the presence of an enzymatically inactive or attenuated molecule that is able to bind to the same site on the target RNA, but is unable to cleave that RNA. In another embodiment, inhibition or down-regulation with antisense oligonucleotides is preferably below that level observed in the presence of, for example, an oligonucleotide with scrambled sequence or with mismatches. In another embodiment, inhibition or down-regulation of gliomedin with the nucleic acid molecule of the instant invention is greater in the presence of the nucleic acid molecule than in its absence.

The term “dsRNA” refers to ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving and/or ligation activity to the molecule (e.g. U.S. Pat. No. 4,987,071).

Chemically synthesizing nucleic acid molecules with modifications (base, sugar and/or phosphate) that prevent their degradation by serum ribonucleases can increase their potency (see e.g., International Publication Nos. WO 93/15187 and WO 91/03162; and U.S. Pat. No. 5,334,711, all of which describe various chemical modifications that can be made to the base, phosphate and/or sugar moieties of the nucleic acid molecules herein).

Examples known in the art describing sugar, base and phosphate modifications that can be introduced into nucleic acid molecules with significant enhancement in their nuclease stability and efficacy include, modification with nuclease resistant groups, for example, 2′-amino, 2′-C-allyl, 2′-flouro, 2′-O-methyl, 2′-H, nucleotide base modifications. Sugar modification of nucleic acid molecules have been extensively described in the art (e.g. International Publication PCT Nos. WO 92/07065; WO 93/15187; WO 97/26270 and WO 98/13526 and U.S. Pat. Nos. 5,334,711; 5,716,824 and 5,627,053; all of which are hereby incorporated in their totality by reference herein). Such publications describe general methods and strategies to determine the location of incorporation of sugar, base and/or phosphate modifications and the like without inhibiting catalysis. In view of such teachings, similar modifications can be used as described herein to modify the siRNA of the instant invention.

While chemical modification of oligonucleotide internucleotide linkages with phosphorothioate, phosphorothioate, and/or 5′-methylphosphonate linkages improves stability, too many of these modifications can cause some toxicity. Therefore when designing siRNA molecules, the amount of internucleotide linkages should be minimized. The reduction in the concentration of these linkages should lower toxicity resulting in increased efficacy and higher specificity of these molecules.

The present invention provides a method of down-regulating gliomedin expression in a cell, comprising contacting the cell with the siRNA molecules of the invention, under conditions suitable for down-regulating of gliomedin expression. According to one embodiment, the suitable conditions include but not limited to the presence of a divalent cation, for example Mg²+.

Methods for the delivery of siRNA molecules are described in Akhtar et al., 1992, Trends Cell Bio., 2, 139; and Delivery Strategies for Antisense Oligonucleotide Therapeutics, ed. Akhtar, 1995 both incorporated herein by reference. WO 94/02595 further describes the general methods for delivery of siRNA molecules. siRNA molecules can be administered to cells by a variety of methods known to those familiar to the art, including, but not restricted to, encapsulation in liposomes, by iontophoresis, or by a incorporation into other vehicles, such as hydrogels, cyclodextrins, biodegradable nanocapsules and bioadhesive microspheres. Alternatively, the nucleic acid/vehicle combination is locally delivered by direct injection or by use of an infusion pump. More detailed descriptions of nucleic acid delivery and administration are provided in WO93/23569; WO99/05094, and WO99/04819 all of which have been incorporated by reference herein.

The present invention further provides pharmaceutical composition comprising the siRNA molecules of the invention in a pharmaceutically acceptable carrier. A pharmacological composition or formulation refers to a composition or formulation in a form suitable for administration, e.g., systemic administration, into a cell or subject, preferably a human. Suitable forms, in part, depend upon the use or the route of entry, for example oral, transdermal or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

The present invention further provides methods of administering to a cell, such as mammalian cell (e.g. human cell), where the cell can be in culture or in a mammal, such as a human, the siRNA molecules of the invention. The nucleic acid-based inhibitors of the invention are added directly, or can be complexed with cationic lipids, packaged within liposomes, or otherwise delivered to target cells or tissues.

The siRNA can be locally administered to relevant tissues ex vivo, or in vivo through injection or infusion pump, with or without their incorporation in biopolymers.

The siRNA molecules of the invention may be expressed from transcription units inserted into DNA or RNA vectors. The recombinant vectors are preferably DNA plasmids or viral vectors. The expressing viral vectors can be constructed based on, but not limited to, adeno-associated virus, retrovirus, adenovirus, or alphavirus. Preferably, the recombinant vectors are capable of delivering and expressing the siRNA molecules in target cells. Alternatively, viral vectors can be used that provide for transient expression of the siRNA molecules. Such vectors can be repeatedly administered as necessary. Once expressed, the siRNA molecules bind to the target RNA and down-regulate its function and/or expression. Delivery of the expressing vectors can be systemic, such as by intravenous or intramuscular administration, by administration to target cells explanted from the patient or subject followed by reintroduction into the patient or subject, or by any other means that would allow for introduction into the desired target cell.

By “vectors” is meant any nucleic acid- and/or viral-based technique used to deliver a desired nucleic acid.

By “subject” is meant an organism, which is a donor or recipient of explanted cells or the cells themselves. The term “subject” also refers to an organism to which the siRNA molecules of the invention can be administered. Preferably, a subject is a mammal or mammalian cells. More preferably, a subject is a human or human cells.

The siRNA molecules of the instant invention, individually, or in combination or in conjunction with other drugs, can be used to treat any diseases or conditions which respond to the attenuation of gliomedin expression.

### Methods of Treatment

The present invention provides several methods of treating neurological damage in a subject in need thereof. The neurological damages that may be treated using the methods of the invention are any one of the methods selected from the group consisting of: multiple sclerosis, axonal injury, lack of ion channel in axolemma, disordered axonal clustering of Na⁺ channels, damaged initial Schwann cell myelination, damaged initial nerve development and demyelination-associated disease.

The demyelination-associated disease may be any one of the following: diabetic neuropathy, Guillain-Barre Syndrome, chronic demyelinating disease, acute demyelinating polyneuropathy and human immunodeficiency viral demyelinating neuropathy, demyelination caused by trauma, inherited neuropathies referred to as Charcot-Marie-Thooth disease (CMT), hereditary motor syndrome and sensory neuropathy (HMSN).

According to one embodiment, the method of treatment comprises administering to the subject a pharmaceutical composition comprising a therapeutically effective amount of gliomedin or an active fragment thereof. Administering is typically carried out by oral administration, intravenous injection, intramuscular injection or intrathecal injection.

For pharmaceutical use, the proteins of the present invention are formulated for parenteral, particularly intravenous or subcutaneous, delivery according to conventional methods. Intravenous administration will be by bolus injection or infusion over a typical period of one to several hours. As detailed above, pharmaceutical formulations will include gliomedin or an active fragment thereof in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., (Mack Publishing Co., Easton, Pa., 19th ed., 1995). Therapeutic doses will generally be in the range of 0.1 to 100 μg/kg of patient weight per day, preferably 0.5-20 μg/kg per day, with the exact dose determined by the clinician according to accepted standards determination of dose is within the level of ordinary skill in the art. The proteins may be administered for acute treatment, over one week or less, often over a period of one to three days or may be used in chronic treatment, over several months or years.

U.S. Pat. No. 6,613,332 provides an orally administrable therapeutic protein by combining the therapeutic protein with a stabilizing agent in an aqueous solution. The solution is coated onto nonpareils and microencapsulated with a water emulsifiable enteric coating composition. The microcapsules are orally administered. The coating protects the protein as it passes through the stomach. Upon reaching the small intestines, the basic pH of the intestinal juices will dissolve the coating, allowing the protein to be released and induce antigen specific immune response which has the specificity of the native molecule. The stabilizing agent protects the therapeutic protein from denaturation during the encapsulation process.

According to another embodiment, the method of treatment comprises transplanting the host cells of the invention in a subject in need thereof. The transplanted host cells may be autologous cells genetically modified to express gliomedin or an active fragment thereof. Preferably, the host cells are transplanted at or near at least one predetermined locus. Optionally, the at least one predetermined locus lacks sufficient levels of Na⁺ channels.

It is possible to carry out the methods of the invention by autologous transplantation. Cells are removed from the body and the vector is introduced thereto as a naked DNA plasmid to form transformed cells which are then re-implanted into the body. Naked DNA vector for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter.

According to yet another embodiment, the method of treatment comprises introducing the expression vectors of the invention to the subject by means of gene therapy. In one embodiment, in the vector is a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes virus 1 (HSV1) vector, an attenuated adenovirus vector, such as the vector described by Stratford-Perricaudet et al., J. Clin. Invest. 90:626-630 (1992), and a defective adeno-associated virus vector.

In another embodiment, the vector is a retroviral vector, e.g., as described in Anderson et al., U.S. Pat. No. 5,399,346; U.S. Pat. No. 4,650,764; 4,980,289; U.S. Pat. No. 5,124,263; and International Patent Publication No. WO 95/07358.

The vector can be introduced by lipofection in vivo using liposomes. Synthetic cationic lipids can be used to prepare liposomes for in vivo transfection of a gene encoding a marker. The use of lipofection to introduce exogenous genes into specific organs in vivo has certain practical advantages. Molecular targeting of liposomes to specific cells represents one area of benefit. It is clear that directing transfection to particular cells represents one area of benefit. It is clear that directing transfection to particular cell types would be particularly advantageous in a tissue with cellular heterogeneity, such as the pancreas, liver, kidney, and brain. Lipids may be chemically coupled to other molecules for the purpose of targeting. Targeted peptides, e.g., hormones or neurotransmitters, and proteins such as antibodies, or non-peptide molecules could be coupled to liposomes chemically.

The methods of the invention may further comprise administering of enhancement agents directed to enhance the effect of gliomedin. Such agents include for example thrombopoietin, thyroid hormone as disclosed in U.S. Pat. No. 6,776,984.

Diagnostic Assays

The antibodies of the invention can be used to detect or image localization of the gliomedin in a subject for the purpose of detecting, diagnosing or monitoring a neurological defect. Such antibodies can be polyclonal or monoclonal, or prepared by molecular biology techniques.

Antibodies can be labeled with a variety of detectable labels including, but not limited to, radioisotopes, ultrasound contrast agents and paramagnetic metals. Targetable diagnostic and/or therapeutically active agents, particularly ultrasound contrast agents are disclosed in U.S. Pat. No. 6,680,047.

In general labeling moieties should be compatible with the detection method that is applied. Thus, for positron emission tomography (PET) radionuclides are typically used. The radionuclide of choice may be selected from F-18, Mn-51, Mn-52m, Fe-52, Co-55, Cu-62, Cu-64, Ga-68, As-72, Br-75, Br-76, Rb-82m, Sr-83, Y-86, Zr-89, Tc-94m, In-110, I-120, and I-124.

In magnetic resonance imaging techniques the agent should comprises a suitable metal, optionally the metal is associated with a chelating agent such as DTPA and DOTA and the like. Metals that can be used as the diagnostic moiety for detection with MRI techniques are selected from the group consisting of gadolinium, manganese, iron, chromium, copper, cobalt, nickel, dysprosium, rhenium, europium, terbium, holmium and neodymium.

For gamma-ray detection the diagnostic moiety bound to the antibody of the invention may be any one or more of the following: Cr-51, Co-57, Co-58, Fe-59, Cu-67, Ga-67, Se-75, Ru-97, Tc-99m, In-111, In-114m, I-123, I-125, I-131, Yb-169, Hg-197, and Tl-201.

Thus, for instance, a diagnostic assay in accordance with the invention for measuring levels of gliomedin compared to normal control bodily fluids, cells, or tissue samples may be used to diagnose the neurological damage.

Those skilled in the art will appreciate that the foregoing embodiments utilize generally cylindrical configurations, but that other shapes are within the scope of the present invention. Thus, the inlet and/or the first and/or second sections could have polygonal cross-section, in which case the limitations discussed above with respect to the dimensions of the outside diameters would instead apply to the outside perimeters.

### EXAMPLES

### Experimental Procedures

Tissue culture methods—Isolated primary neonatal rat Schwann cell were maintained on Primeria plates (Falcon) in DME containing 3% FCS, 10% conditioned medium of CHO cells expressing NDF/Neuregulin) (Peles et al., 1992, Cell 69, 205-216), and 4 μM forskolin (Sigma). For binding and staining experiments, Schwann cells were grown on poly L lysine-coated 12-mm coverslips. Dissociated rat DRG cultures were grown in Neurobasal medium (Gibco) supplemented with B27 (Gibco) and 50 ng/ml NGF (Alomone labs) (NB medium), or C medium (Einheber, Peles et al., J Cell Biol 139, 1495-1506, 1997), for five days before used in binding experiments. Purified DRG neurons were established by treating dissociated mixed cultures with two cycles (two days each) of NB medium containing 10 μM Uridine{tilde under (/)}.1 μM 5′-Fluoro 2′-deoxyuridine (Sigma) to eliminate fibroblasts and Schwann cells. Myelinating DRG explants were prepared by culturing three E16 DRGs/slide on coverslips precoated with 0.4 μg/ml Matrigel (BD Biosciences) and 10 μg/ml Poly-D-lysine (Sigma). For dissociated explants, 20,000 cells/slide were plated on 10 μg/ml laminin coated coverslips. Cultures were maintained for two days in NB medium and then switched to Basal Eagle's Medium (BME; Gibco) containing ITS supplements (Sigma), 0.2% BSA, 4 g/l D-glucose and 50 ng/ml NGF (BN medium). Myelination was induced after an additional 10 days by the addition of 15% heat inactivated FCS (Gibco) and 50 μg/ml L-ascorbic acid (Sigma) (BNC medium). Viral infection was carried out for two hours with 8 μg/ml polybrene on the second, third and fourth days after plating with undiluted viral stock; myelination was induced six days later and proceeded for additional 12 days before analysis.

Expression cloning—Construction of rat Schwann cell cDNA library and expression cloning was done essentially as described previously (Peles et al., 1995, Cell 82, 251-260). Briefly, library pools each containing 750 independent plasmid DNAs were prepared and transfected into COS7 cells using Lipofectamine (Gibco). After 48 hours the cells were incubated with medium containing NF155Fc that was premixed with biotinylated goat-anti-human antibody (Jackson Laboratories). Unbound fusion proteins were washed out and the cells were fixed with 4% paraformaldehyde. Cells were then incubated with streptavidin-alkaline phosphatase for 30 min at 23° C. followed by the addition of BCIP-NBT alkaline phosphates substrate (Vector Laboratories). One positive pool was subdivided and re-screened three additional times until a single clone (F10) was isolated. DNA sequencing of the isolated clone was performed on both strands by priming with specific oligonucleotide primers (GenBank accession number AY266116). Protein topology and the location of the transmembrane domain were predicted using SOSUI-Classification and Secondary Structure Prediction of Membrane Proteins (http://sosui.proteome.bio.tuat.ac.jp/sosuiframe0.html).

Constructs and antibodies—Fc-fusions containing the collagen repeats (COL, residues 49-294) and the olfactomedin domain (OLF, residues 277-547) were made by cloning the corresponding DNA to pSecTagA vector (Invitrogen), which contains the signal sequence of the κappa chain of human IgG and then transferred to pCX-Fc (Gollan, Peles et al., 2003, J Cell Biol 163, 1213-1218). NF186Fc, L1-Fc and NrCAM-Fc were obtained from Martin Grumet. Contactin-Fc was described previously (Peles et al., 1995; siRNA-constructs for rat Gliomedin (target-sequence RNAi#1 TGAGCGCCATTCTCCACAA-SEQ ID NO:3, target-sequence RNAi#3 ACAACTCTCTCTACTACCA-SEQ ID NO:4) and rat MAG (target-sequence CATGGCGTCTGGTATTTCA-SEQ ID NO:5) were cloned into a modified pRETRO-SUPER vector in which the puromycin resistance gene was replaced with an EGFP (Clontech). Retroviral stocks were prepared using the Helper-virus-free Phoenix-Eco packaging cells (kindly provided by G. Nolan, Stanford, Calif.). Polyclonal antibodies to gliomedin were generated by immunizing rabbits with synthetic peptides corresponding to amino acid residues 273-287 (CVIPNDDTLVGRA-SEQ ID NO:6; Ab 7202) present in the extracellular region of rat gliomedin. Monoclonal MAb94 antibody to gliomedin (ATCC Accession No.______) was generated by immunizing mice with the above extracellular-containing peptide as described previously (Poliak et al., 1999, Neuron 24, 1037-1047). Rabbit polyclonal and mouse monoclonal antibodies to Caspr (Peles et al., 1997, Embo J 16, 978-988; Poliak, Peles et al., 1999, ibid), mouse ant-Na⁺ channels (Rasband, Peles et al., 1999, J Neurosci 19, 7516-7528), chick anti-betaIV spectrin, and rabbit anti-NrCAM were previously described. Pan-neurofascin monoclonal antibody was obtained from Matthew Rasband and rabbit anti-NF186 antibody from Peter Brophy. Other primary antibodies, including rat anti-MBP (Chemicon), mouse anti-neurofilaments (Sigma), mouse anti-MAG (clone 513; Roche), and mouse anti-ankyrin G (Santa Cruz) were obtained from commercial sources. Cy3, Cy5 and Alexa-conjugated secondary antibodies were purchased from Jackson laboratory and Molecular Probes, respectively.

RNA expression analysis—Northern blot analysis of multiple human tissues RNA samples (BD Bioscience) was performed as previously described in Spiegel, Peles et al., (Mol Cell Neurosci 20, 283-297, 2002) using a 1 kb fragment of human gliomedin, which was amplified from Hs683 human glioblastoma cDNA. For rat tissues, a blot containing total RNA from sciatic nerve and brain was hybridized to a 964 nucleotide BglI/EcoRI DNA fragment of rat gliomedin or β-actin probes. In situ hybridization was performed using cRNA probe containing the entire gliomedin cDNA as previously described (Spiegel, Peles et al., ibid).

Immunofluorescence—Teased sciatic nerves and frozen sections were prepared as previously described (Poliak, Peles et al., 2002, J Cell Biol 159, 361-372; Poliak, Peles et al., 2003, Nat Rev Neurosci 4, 968-980). Briefly, samples were postfixed/permeabilzed in cold methanol for 20 min in ±20° C., or permeabilzed in PBS containing 0.5% Triton X-100 for 10 min. Slides were blocked for 30 min with PBS containing 10% normal goat serum, 0.1% Triton X-100 and 1% glycine (blocking solution). The samples were incubated overnight at 4° C. with primary antibodies diluted in blocking solution; washed with PBS; incubated for 1 hour at 23° C. with secondary anti-mouse-Cy3 (Jackson Laboratories) and anti-rabbit-488 (Molecular Probes). Slides were then mounted with elvanol and analyzed using a Nikon Axioplan microscope or a BioRad confocal microscope. Triple labeling of myelinated cultures, was done using different primary antibodies followed by anti mouse or chick-Cy3, anti rabbit-Alexa-488, and anti-rat-Cy5.

Immunoelectron microscopy—Sciatic nerves were fixed for 30 min in 0.1M cacodylate buffer containing 3% paraformaldehyde, 1% glutaraldehyde, 3% sucrose, 5 mM CaCl₂. Briefly, fixed tissue was embedded in 7% agar noble, sectioned on a vibrotome, infiltrated in LR Gold and embedded in gelatine capsules, followed by UV polymerization. Tissue sections on formvar-coated nickel grids were blocked with 0.5 BSA, 0.5% gelatine from porcine skin 0.1% Tween-20 in PBS and incubated with affinity purified anti-gliomedin antibody in the same buffer for 2 h in room temperature. Grids were washed seven times in PBS and incubated for 1 h with goat anti rabbit IgG conjugated to 10 nm gold (1:20; Aurion). Grids were postfixed in 2% aqueous OsO4, stained with 2% uranyl acetate and Reynolds's lead citrate, and examined using a Philips CM-12 transmission electron microscope.

Fc-fusion binding, clustering and perturbation experiments—Transfected COS7 cells, isolated Schwann cells or dissociated DRG explants or purified DRG neurons grown on glass slides were washed once in DMEM and incubated for 25 min at 23° C. with medium containing different Fc-fusions proteins that were already incubated with anti human Fc-Cy3 (Jackson Laboratories). After two washes with DMEM and an additional wash in PBS, the cultures were fixed with 4% paraformaldehyde for 10 (cells) or 15 (DRG explants) minutes. The fixed slides were then labeled with various antibodies or directly mounted with elvanol. For clustering experiments, DRG neurons were grown for 12-13 days on slides coated with 10 μg/ml Poly-D-lysine (Sigma) and laminin (Sigma) before binding. The neurons were then incubated with medium containing OLF-Fc, GLDN-Fc, or IgSF4-Fc as described above, washed once with Neurobasal medium and grown for additional 48-72 hours in NB before fixing and staining. Quantification was done from two 72 h and one-48 h experiments by counting the number of OLF-FC or IgSF4-Fc sites (1.5-4.5 μm) containing Na⁺ channels, neurofascin, ankyrin G or βIV spectrin. For Fc perturbation experiments, 100 mg/ml purified proteins were added to DRG explants with the induction of myelination and was replaced every second day. The cultures were fixed and stained after additional 12-15 days.

### Example 1

### Schwann Cells Exhibit Specific Binding Sites for Soluble Neurofascin but not for Neurones

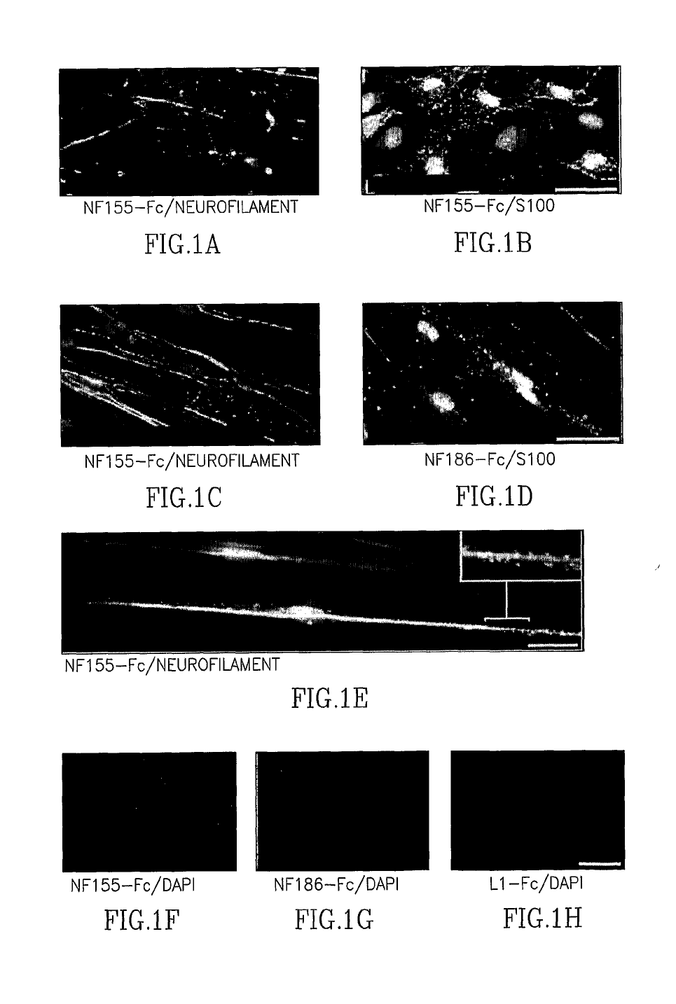

The presence of neurofascin at the nodal axolemma suggests that it may interact with a glial receptor found at the adjacent Schwann cell membrane. To test this possibility, we used the extracellular domain of either the 186-kDa or 155-kDa isoforms of neurofascin fused to human Fc (NF155-Fc, NF186-Fc) in binding experiments on mixed dorsal root ganglia (DRG) cultures. Both neurofascin isoforms bound to most, but not all, Schwann cells in the culture and bound exclusively to S100-positive Schwann cells and not to neurofilament-labeled axons (FIG. 1A-D). Neurofascin binding was occasionally detected on Schwann cells at the initial stage of axon enwrapping (FIG. 1E). Similarly, NF155-Fc and NF186-Fc also bound to primary cultured rat Schwann cells (FIG. 1F-G). In contrast, Schwann cells did not bind the extracellular domain of several related IgCAMs, such as L1 (FIG. 1H) or contactin. These results suggest that Schwann cells express a specific ligand for neurofascin.

### Example 2

### Identification of a Schwann Cell Ligand for Neurofascin

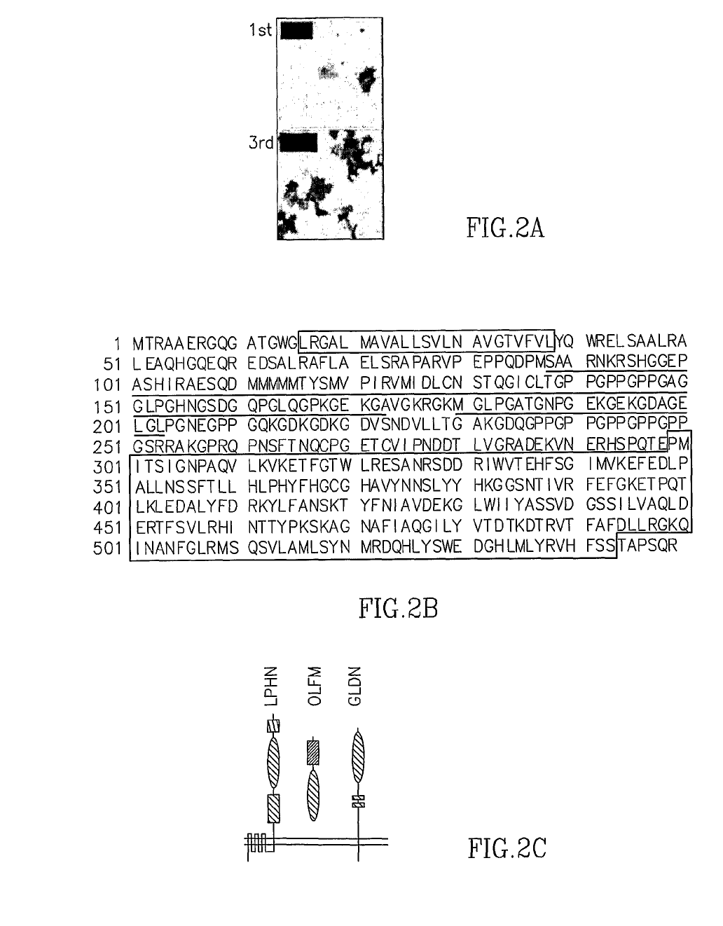

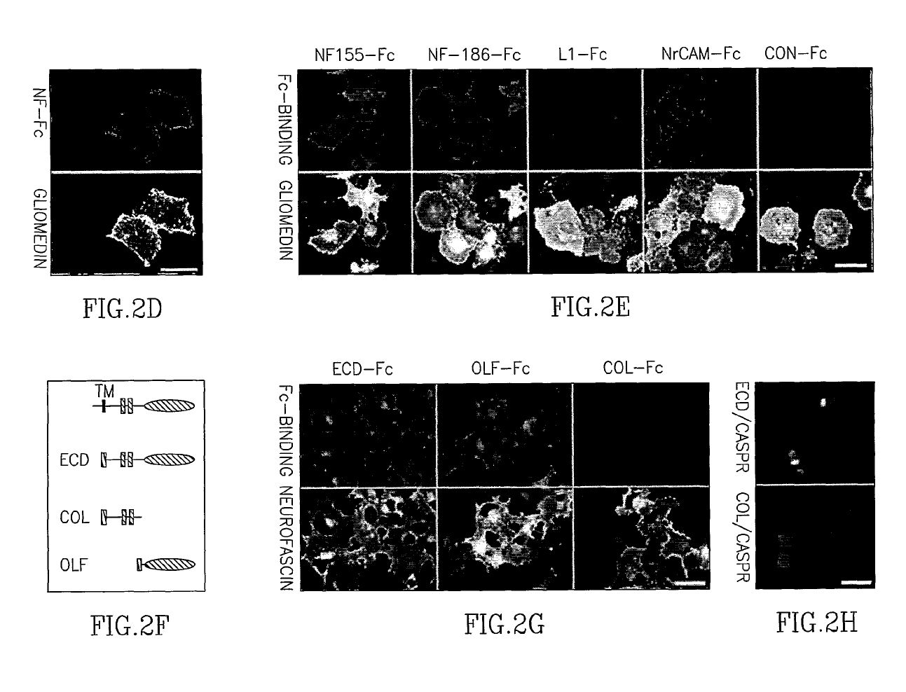

For identification of gliomedin as a glial ligand for the nodal IgCAMs, a rat Schwann cell cDNA library was constructed and screened by expression cloning in order to isolate the putative glial ligand for neurofascin. Plasmid pools made from this cDNA library were transfected into COS7 cells, which were subsequently screened for their ability to bind NF-Fc, as previously described (Peles, 1995; ibid). One of six positive pools was further subdivided into smaller pools and re-screened until a single plasmid was isolated (FIG. 2A; SEQ ID NO:1). Sequence analysis of this cDNA clone revealed a single open reading frame of 1647 nucleotides, which encodes for a 549 amino acid protein (FIG. 2B and Table 1).

TABLE 1

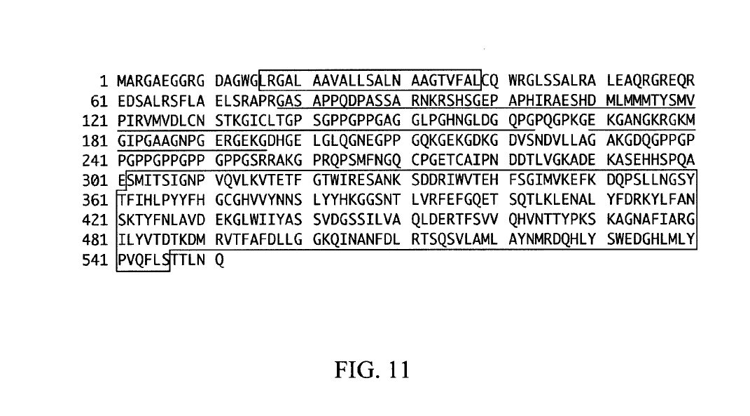

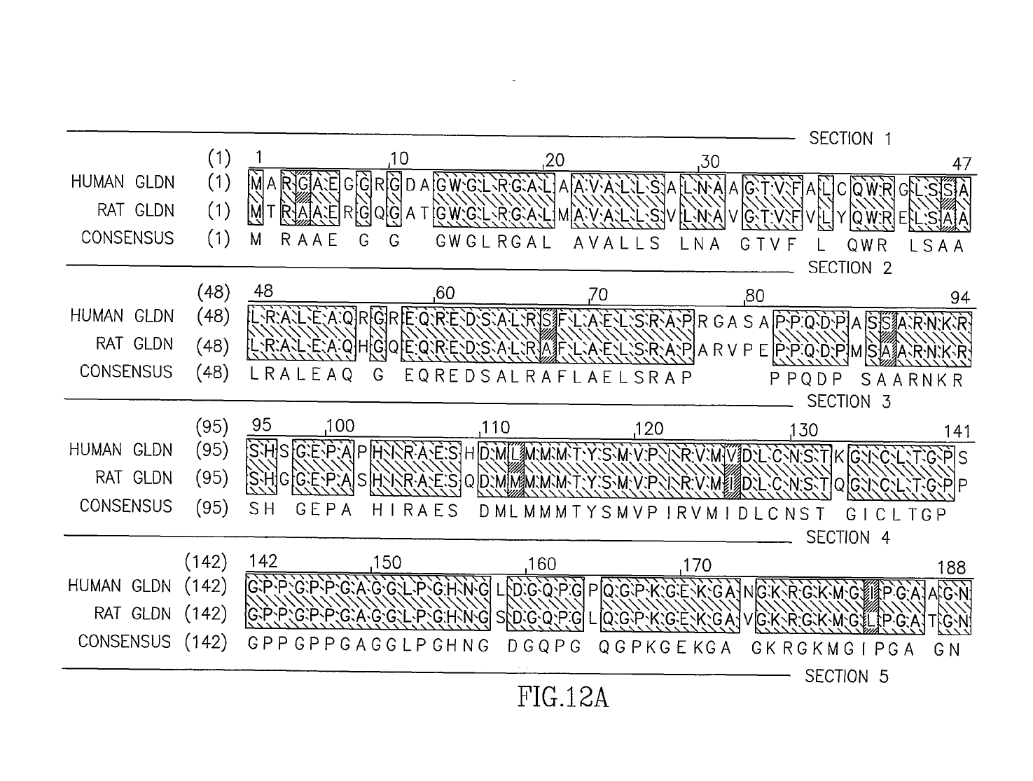

Amino acid and nucleotide sequences of fragments of rat gliomedin

| SEQ | | |
|---|---|---|
| ID | | |
| NO: | Description | Sequence |
| 8 | rat | TGPPGPPGPPGAGGLPGHNGSDGQPGLQGPKGEKGAVGKRGKMGLPGATGNPGEKGEKGDAG |
| | gliomedin | ELGLPGNEGPPGQKGDKGDKGDVSNDVLLTGAKGDQGPPGPPGPPGPPGPPGSR |
| | collagen GXY | |
| | repeats | |
| | domain with | |
| | interruption | |
| | (FIG. 2B) | |
| 9 | rat | PMITSIGNPAQVLKVKETFGTWLRESANRSDDRIWVTEHFSGIMVKEFEDLPALLNSSFTLL |
| | gliomedin | HLPHYFHGCGHAVYNNSLYYHKGGSNTIVRFEFGKETPQTLKLEDALYFDRKYLFANSKTYF |
| | OLF domain | NIAVDEKGLWIIYASSVDGSSILVAQLDERTFSVLQHINTTYPKSKAGNAFIAQGILYVTDT |
| | (FIG. 2B) | KDTRVTFAFDLLRGKQINANFGLRMSQSVLAMLSYNMRDQHLYSWEDGHLMLYPVHFSS |
| 10 | the entire | YQWRELSAALRALEAQHGREQREDSALRAFLAELSRAPARVPEPPQDPMSAARNKRSHGGEP |
| | rat | ASHIRAESQDMMMMMTYSMVPIRVMIDLCNSTQGICLTGPPGPPGPPGAGGLPGHNGSDGQP |
| | gliomedin | GLQGPKGEKGAVGKRGKMGLPGATGNPGEKGEKGDAGELGLPGNEGPPGQKGDKGDKGDVSN |
| | extra- | DVLLTGAKGDQGPPGPPGPPGPPGPPGSRRAKGPRQPNSFTNQCPGETCVIPNDDTLVGRAD |
| | cellular | EKVNERHSPQTEPMITSIGNPAQVLKVLETFGTWLRESANRSDDRIWVTEHFSGIMVKEFED |
| | domain | LPALLNSSFTLLHLPHYFHGCGHAVYNNSLYYHKGGSNTIVRFEFGKETPQTLKLEDALYFD |
| | (FIG. 2B) | RKYLFANSKTYFNIAVDEKGLWIIYASSVDGSSILVAQLDERTFSVLQHINTTYPKSKAGNA |
| | | FIAQGILYVTDTKDTRVTFAFDLLRGKQINANFGLRMSQSVLAMLSYNMRDQHLYSWEDGHL |
| | | MLYPVHFSSTAPSQR |
| 11 | cDNA of the | TACCAGTGGCGCGAGCTGAGCGCGGCGCTGCGGGCACTGGAGGCGCAACACGGCCGGGAGCA |
| | entire rat | GCGCGAGGACAGCGCCCTACGCGCCTTTCTAGCTGAATTAAGTCGTGCGCCAGCCCGAGTCC |
| | gliomedin | CCGAACCACCCCAGGACCCCATGAGTGCAGCGCGCAATAAGCGCAGCCACGGCGGCGAGCCT |
| | extra- | GCGTCACACATCCGCGCCGAGAGCCAGGACATGATGATGATGATGACCTACTCCATGGTGCC |
| | cellular | GATCCGGGTGATGATAGACCTGTGCAACAGCACCCAGGGCATCTGCCTTACAGGACCACCGG |
| | domain | GCCCACCAGGACCTCCAGGAGCTGGTGGGTTACCAGGCCACAATGGATCAGATGGACAGCCT |
| | | GGTCTCCAGGGCCCAAAAGGAGAAAAAGGAGCAGTTGGGAAGAGAGGAAAAATGGGGTTACC |
| | | CGGAGCCACAGGAAATCCAGGGGAAAAGGGAGAGAAGGGAGATGCTGGTGAACTGGGCCTAC |
| | | CTGGAAATGAGGGACCACCAGGACAGAAAGGAGACAAAGGAGACAAAGGAGATGTGTCCAAT |
| | | GACGTGCTTTTGACAGGTGCCAAAGGTGACCAAGGGCCCCCTGGCCCACCTGGACCCCCAGG |
| | | GCCTCCAGGCCCTCCTCGAAGCAGAAGAGCCAAAGGCCCTCGGCAGCCAAATTCGTTCACCA |
| | | ACCAGTGTCCAGGGGAGACGTGTGTCATACCCAATGATGATACCTTGGTGGGGAGAGCTGAT |
| | | GAGAAAGTCAATGAGCGCCATTCTCCACAAACAGAACCCATGATCACGTCCATTGGTAACCC |
| | | GGCCCAAGTCCTCAAAGTGAAAGAGACTTTTGGGACCTGGCTAAGAGAGTCTGCTAACAGGA |
| | | GTGATGACCGCATTTGGGTGACTGAACATTTTTCAGGCATCATGGTGAAGGAGTTTGAAGAC |
| | | CTGCCCGCCCTCCTGAATAGCAGCTTCACCCTCCTCCACCTCCCACATTACTTCCATGGCTG |
| | | CGGGCACGCTGTTTACAACAACTCTCTCTACTACCACAAAGGAGGCTCCAACACCATAGTGA |
| | | GATTTGAATTTGGGAAAGAGACACCTCAAACTCTGAAGCTTGAAGATGCTTTGTATTTTGAT |
| | | CGAAAATACCTCTTTGCAAATTCCAAGACTTACTTCAACATAGCAGTGGATGAGAAGGGCCT |
| | | CTGGATTATCTACG |
| | | CCTCGAGTGTGGATGGCTCAAGCATCCTTGTGGCACAGCTGGACGAGAGGACATTCTCTGTG |
| | | CTGCAGCACATCAATACCACATACCCCAAGTCCAAGGCTGGCAATGCCTTCATAGCTCAAGG |
| | | GATCCTCTATGTCACGGACACCAAAGATACAAGGGTCACGTTTGCCTTTGATTTGTTACGAG |
| | | GGAAGCAGATCAATGCAAACTTCGGTCTCAGAATGTCACAGTCTGTTCTTGCCATGTTGTCA |
| | | TACAATATGAGAGACCAGCATTTGTACTCGTGGGAAGACGGCCACCTGATGCTCTATCCTGT |
| | | GCACTTTTCGTCAACAGCACCCAGCCAGCGATAG |
| 12 | cDNA of rat | CTGGGCCTACCTGGAAATGAGGGACCACCAGGACAGAAAGGAGACAAAGGAGACAAAGGAGA |
| | gliomedin | TGTGTCCAATGACGTGCTTTTGACAGGTGCCAAAGGTGACCAAGGGCCCCCTGGCCCACCTG |
| | OLF domain | GACCCCCAGGGCCTCCAGGCCCTCCTGGAAGCAGAAGAGCCAAAGGCCCTCGGCAGCCAAAT |
| | | TCGTTCACCAACCAGTGTCCAGGGGAGACGTGTGTCATACCCAATGATGATACCTTGGTGGG |
| | | GAGAGCTGATGAGAAAGTCAATGAGCGCCATTCTCCACAAACAGAACCCATGATCACGTCCA |
| | | TTGGTAACCCGGCCCAAGTCCTCAAAGTGAAAGAGACTTTTGGGACCTGGCTAAGAGAGTCT |
| | | GCTAACAGGAGTGATGACCGCATTTGGGTGACTGAACATTTTTCAGGCATCATGGTGAAGGA |
| | | GTTTGAAGACCTGCCCGCCCTCCTGAATAGCAGCTTCACCCTCCTCCACCTCCCACATTACT |
| | | TCCATGGCTGCGGGCACcCTGTTTACAACAACTCTCTCTACTACCACAAAGGAGGCTCCAAC |
| | | ACCATAGTGAGATTTGAATTTGGGAAAGAGACACCTCAAACTCTGAAGCTTGAAGATGCTTT |
| | | GTATTTTGATCGAAAATACCTCTTTGCAAATTCCAAGACTTACTTCAACATAGCAGTGGATG |
| | | AGAAGGGCCTCTGGATTATCTACGCCTCGAGTGTGGATGGCTCAAGCATCCTTGTGGCACAG |
| | | CTGGACGAGAGGACATTCTCTGTGCTGCAGCACATCAATACCACATACCCCAAGTCCAAGGC |
| | | TGGCAATGCCTTCATAGCTCAAGGGATCCTCTATGTCACGGACACCAAAGATACAAGGGTCA |
| | | CGTTTGCCTTT |
| 13 | cDNA of rat | ACAGGACCACCGGGCCCACCAGGACCTCCAGGAGCTGGTGGGTTACCAGGCCACAATGGATC |
| | gliomedin | AGATGGACAGCCTGGTCTCCAGGGCCCAAAAGGAGAAAAAGGAGCAGTTGGGAAGAGAGGAA |
| | collagen | AATGGGGTTACCCGGAGCCACAGGAAATCCAGGGGAAAAGGGAGAGAAG |
| | repeat | |
| | domain | |
| 14 | human | MARGAEGGRGDAGWGLRGALAAVALLSALNAAGTVFALCQWRGLSSALRALEAQRGREQRED |
| | gliomedin | SALRSFLAELSRAPRGASAPPQDPASSARNKRSHSGEPAPHIRAESHDMLMMMTYSMVPIRV |
| | | MVDLCNSTKGICLTGPSGPPGPPGAGGLPGHNGLDGQPGPQGPKGEKGANGKRGKMGIPGAA |
| | | GNPGERGEKGDHGELGLQGNEGPPGQKGEKGDKGDVSNDVLLAGAKGDQGPPGPPGPPGPPG |
| | | PPGPPGSRRAKGPRQPSMFNGQCPGETCAIPNDDTLVGKADEKASEHHSPQAESMITSIGNP |
| | | VQVLKVTETFGTWIRESANKSDDRIWVTEHFSGIMVKEFKDQPSLLNGSYTFIHLPYYFHGC |
| | | GHVVYNNSLYYHKGGSNTLVRFEFGQETSQTLKLENALYFDRKYLFANSKTYFNLAVDEKGL |
| | | WIIYASSVDGSSILVAQLDERTFSVVQHVNTTYPKSKAGNAFIARGILYVTDTKDNRVTFAF |
| | | DLLGGKQINANFDLRTSQSVLAMLAYNMRDQHLYSWEDGHLMLYPVQFLSTTLNQ |
| 15 | cDNA of | ctctgggctg ctcgggcgcc accactactg tccccatccc gaccagacag |
| | human | ctgggccaaa gggtgacttg actcacgacg ctgccaccag cccacggctt |
| | gliomedin | gcccgaggcg tataaaggct gccagggccg caggctgcca agccctgccc |
| | | tgcccaaggc gcatagagca tggcccgagg cgctgaggga ggccgtgggg |
| | | acgcgggttg gggcctgcgt ggcgccctgg cggccgtggc gctgctctcg |
| | | gcgctcaacg ctgcgggcac ggtgttcgcg ctgtgccagt ggcgcgggct |
| | | gagctcggcg ctgcgggctt tggaggcgca gcggggccgg gagcagcgcg |
| | | aggacagtgc cctgcgctcc ttcctggccg agttgagccg cgcgccgcgc |
| | | ggggcgtccg caccacccca agacccggcc agctcagctc gcaacaagcg |
| | | cagccacagc ggcgagcccg cgccgcatat ccgcgccgag agccatgaca |
| | | tgctgatgat gatgacctac tccatggtgc cgatccgagt gatggtggac |
| | | ctgtgcaaca gcaccaaggg catctgcctc acaggacctt ctggaccacc |
| | | aggacctccg ggagccggcg ggttgccagg acacaacgga ttggatggac |
| | | agcctggtcc tcagggccca aaaggagaaa aaggagcaaa tggaaaaaga |
| | | ggaaaaatgg ggatacctgg agctgcagga aatccagggg aaaggggaga |
| | | aaagggagac catggtgaac tgggcctgca gggaaatgag ggcccaccag |
| | | ggcagaaggg agaaaagggt gacaaaggag atgtgtccaa cgacgtgctc |
| | | ctggcaggtg ccaaaggtga ccaaggccca cccggtccac ctgggccccc |
| | | aggccctcca ggtcctccag ggccccctgg aagcagaaga gccaaaggcc |
| | | ctcggcagcc aagcatgttc aacggccagt gcccaggtga gacttgtgcc |
| | | ataccaaatg atgatacctt ggttggaaaa gctgatgaga aagccagtga |
| | | acaccattcc ccacaagcag aatccatgat cacttccatt ggaaacccag |
| | | tgcaagtact gaaagtgaca gagacatttg ggacttggat aagagagtct |
| | | gctaacaaga gtgatgaccg gatttgggtg acagagcatt tttcaggcat |
| | | catggttaag gaattcaagg atcagccctc acttctgaat ggcagttaca |
| | | cgttcatcca ccttccatac tatttccatg gctgtgggca cgttgtttac |
| | | aacaactctc tctactacca caaagggggt tctaataccc tagtgagatt |
| | | tgaatttggc caggaaacat cccaaactct gaagcttgaa aatgccttgt |
| | | attttgatcg aaaatacctt tttgcaaatt ccaaaactta cttcaatcta |
| | | gctgtagatg aaaagggcct ttggattatc tatgcgtcaa gtgtggacgg |
| | | ctcgagcatt cttgtagcac aactggatga gaggacattc tcagtggtgc |
| | | aacacgtcaa taccacgtac cctaaatcca aggctggcaa cgccttcatt |
| | | gcccgaggaa tcctctatgt cacagacacc aaagatatga gggtcacatt |
| | | tgcctttgat ttgttaggag ggaaacagat caatgcaaac tttgatttaa |
| | | gaacttccca gtctgttctt gccatgttag catacaacat gagagatcag |
| | | catttatatt catgggaaga tggccattta atgctttatc ctgtgcagtt |
| | | tttgtcaact accttaaatc agtgatgtgc tgcattcggc tcccttcagc |
| | | aaatttcagg ggttttctgg gaccagttct cccccaacag gaaacttgtt |
| | | tttttaacgt cagccagata tttagaaaat aacctcaaaa gtgtttatat |
| | | ggtcagtgag ccccgcttag tgaaatagca acagattgga agttgaaatg |
| | | gctgagattt ggtgatctcc ccacagctgg ctctgcaagt tacctctttc |
| | | tccttgggcc ttagtttccc cattggtaat ctgaattggc taagatgatt |
| | | ggggagattt tctgtacctg taggtaattt ggtgattctt ggtggctgct |
| | | cttctcacaa cttttatgta tctgcttctg tcgtttagct tttttagcca |
| | | catgctgacc aaatttacct ttgagttgat aagtccagtg gcttgagtag |
| | | tgaatccctc agtgctgact tatatcttgt tctttgaaaa aatgcattga |
| | | ctctttaaga catctaaagt atcacattat ccataattta ttgcttttct |
| | | ttgcatctgc acctgccacc acagaataac cattaccctc agctgctgat |
| | | tgggcagctc tgagattagc aaaagccagg gacagctaca tgttcaggtt |
| | | tttttttttt tttttttttc aataagctat tttttttctt ttcttatttt |
| | | aaatagagag agagtcttgc tatgtttcca aggctggtct tgaactcctg |
| | | gggctcaagt gatcctcctg ccttggcctc ccaaaatgct ggattacagg |
| | | catgtgtgcc tggcccaggt ttcttaataa aacagaatca tgatcttcca |
| | | ggttcccccc agtttctgat catgttgatt tgtagctgtg gatcatgaac |
| | | actgaatcct cagatcactc tgacttctta tgcttctcct gtggatccac |
| | | tatcaaagta ctaaatgctg tgtaagtaga cgttaatctg gctggaacca |
| | | tgggaagcac ttggcagtgt tcagaagaga ggctccattt gtggctatta |
| | | tgtagaactg ggccagagcc agtccattgc ctgttttttt aaataaggtt |
| | | ttactgagca cagccacact catttgttta tgcagtacgg cctgacattg |
| | | cttttgctct gcaacagcag agtcgagtca ttgcaacaaa gagcatatgg |
| | | ccccacagtg cctaaaatat tgaccagcta cccctttatg gaaaaagatt |
| | | gctgactcat gataaagaat ataaagtgag cctgattctt gaaaaaatca |
| | | gaaccagagc ctgttttgtt ttgttctaaa ctaagaagcc gcataggatg |
| | | tgacttgcgt tttgagtaga ggggaaggct gataacggcg taagatgaag |
| | | tggccctcca caaaggctgg ttaggggaca gttctttcta taacatagtt |
| | | ttaaaggatg tgatctggtc cccttggatg ccaggagaga atccagttga |
| | | acttgctcct aaatgctctt aaatatgcat attttctgcc aactcacttc |
| | | tttaaacatc tttcagccca gcgctgcggc cccgggaagg gccactgcga |
| | | atagagagga agctggaaaa gttcctgggg ctctgcagcc aggaagggga |
| | | accagggcaa atcttatgta aagatttttc agcaacttgt cccaatttgt |
| | | gtgtattctg aaactttctc tttgggacca aattcattct caatggccct |
| | | gagttcaata tattattaac agcagtattt taaaacttag ggttgaactg |
| | | ggcatggtgg cacataactg caatcccagc tactttggag gcagggatgg |
| | | gaggatcact tgaggccagg atctcaggac cagcctagag agatcccatc |
| | | tctaaaaaat aaaatataag aaaataaaac ttaggggata tacagattta |
| | | aatattcaaa tctccctgct cccctgaaag tccccaggca gctgtcaatg |
| | | acttgtttgt tgtgttctca atatgatggc tatttgaaac ttcacctact |
| | | tttcattaga ttggttgtac catgtcacct tagcttttaa aaatactctt |
| | | ttcagattca cgttctctaa caaagagtct catgttcaag atcaatatgt |
| | | ctaataagtg ctggtgtcct tttaaagtat ttaaatatat atgttgctgt |
| | | tgctgaatac aggagaccag gttaggaata tagtttcata ataatagtac |
| | | atacaatact aattgtatat aaggtagcaa ccaaaagagg ttgttaatta |
| | | gcacatattc cttttagaaa aatgtttcag aaacctcagt cttgatatct |
| | | gagctatctg ggctccctta cttgtgagta agggatcatg ctcaccactg |
| | | gagaagctta caccgggact ttttttcttt tttctttttt ttttgctatg |
| | | acagagtaat gctaacgtaa ggacaactga gtttgatcag tgtttaatcg |
| | | cagtgggtaa tcttatctga ttgtctttaa aagtgaaaag gattaagatt |
| | | ttattctttc ttgtaaacat tacttgattt tttaaagaag ttttgggctc |
| | | actgctaaaa tagagtatac aactgaatgt ttttaagtca agatactgtt |
| | | ttaggagttt accctctcat ttataaccaa agttgctcta aaacactttc |
| | | caaatatctg cacttctgat gtcagaatca aaccagataa ttctctaatt |
| | | cttctttaat ctaaagtaga tagcttacca ctggaaagta aacaaaacca |
| | | tccctcccaa cctcaaagct aggacacact ctatttcaag gcattttctt |
| | | tcagctgata aggtgtcctc ctgaagccaa gtaggtggtt ctggtctcca |
| | | agtatcgtta agcacaggtg ctatgacaga aaaagttctg gggtggaagt |
| | | tttaagatga ggagttctga tcttaggcat cttaacagtc acaaggtgaa |
| | | aagtcaaatg aaacagtaca attcttgatg agtgaggtgt catcttccaa |
| | | ccacacagag gacgttttgg ctatgatcat ctgatggcaa gtgaaggaga |
| | | aatgagtgat agggctttgc gttttcatcc agatgctgtg gccctgtgtt |
| | | tcacagcatt aagagccata atttccaacc tgcacagatc ctgaacaaca |
| | | aatgaataac gatgaatgtc tttttggttg taatttaaca |
| | | agtcaaataa aataatcatt gctgagcaca atcacatgtt gaaa |
| 18 | entire | CQWRGLSSALRALEAQRGREQREDSALRSFLAELSRAPRGASAPPQDPASSARNKRSHSGEP |
| | extra- | APHIRAESHDMTMMMTYSMVPIRVMVDLCNSTKGICLTGPSGPPGPPGAGGLPGHNGLDGQP |
| | cellular | GPQGPKGEKGANGKRGKMGIPGAAGNPGERGEKGDHGELGLQGNEGPPGQKGEKGDKGDVSN |
| | domain of | DVLLAGAKGDQGPPGPPGPPGPPGPPGPPGSRRAKGPRQPSMFNGQCPGETCAIPNDDTLVG |
| | human | KADEKASEHHSPQAESMITSIGNPVQVLKVTETFGTWIRESANKSDDRIWVTEHFSGIMVKE |
| | gliomedin | FKDQPSLLNGSYTFIHLPYYFHGCGHVVYNNSLYYHKGGSNTLVRFEFGQETSQTLKLENAL |
| | (FIG. 11) | YFDRKYLFANSKTYFNLAVDEKGLWIIYASSVDGSSILVAQLDERTFSVVQHVNTTYPKSKA |
| | | GNAFIARGILYVTDTKDMRVTFAFDLLGGKQINANFDLRTSQSVLAMLAYNMRDQHLYSWED |
| | | GHLMLYPVQFLSTTLNQ |
| 19 | human | SMITSIGNPVQVLKVTETFGTWIRESANKSDDRIWVTEHFSGIMVKEFKDQPSLLNGSYTFI |
| | gliomedin | HLPYYFHGCGHVVYNNSLYYHKGGSNTLVRFEFGQETSQTLKLENALYFDRKYLFANSKTYF |
| | OLF domain | NLAVDEKGLWIIYASSVDGSSILVAQLDERTFSVVQHVNTTYPKSKAGNAFIARGILYVTDT |
| | (FIG. 11) | KDMRVTFAFDLLGGKQINANFDLRTSQSVLAMLAYNMRDQHLYSWEDGHLMLYPVQFLS |
| 20 | human | TGPSGPPGPPGAGGLPGHNGLDGQPGPQGPKGEKGANGKRGKMGIPGAAGNPGERGEKGDHG |
| | gliomedin | ELGLQGNEGPPGQKGEKGDKGDVSNDVLLAGAKGDQGPPGPPGPPGPPGPPGPPGSR |
| | collagen | |
| | GXY repeats | |
| | domain | |
| | (FIG. 11) | |

The predicted polypeptide has the hallmarks of a type II transmembrane protein, containing a short (15 aa) cytoplasmic tail at its amino terminus and a carboxy-terminal extracellular region, which includes a collagen triple helix repeat (COL) and a single olfactomedin domain (OLF; FIG. 2B-C). The collagen domain of gliomedin consists of 28 GXY repeats, wherein G corresponds to Glyceine and X or Y corresponds to any amino acid, an interruption of seven amino acids, namely SNDVLLT in rat and mouse gliomedin (SEQ ID NO:16) and SNDVLLA in human (SEQ ID NO:17), followed by eight additional GXY repeats in mouse and rat and nine in human (FIG. 2B and Table 1). This protein, which was termed by the inventors of the present invention gliomedin, belongs to a growing family of olfactomedin-related molecules (Loria et al., ibid). A sequence corresponding to the extracellular domain of mouse gliomedin was also identified as a liver cancer related gene (CRG-L2; Graveel et al., 2003; ibid). The presence of both OLF and COL domains in the extracellular region of gliomedin places it within a distinct subgroup of the olfactomedin proteins, recently termed Colmedins (Loria et al., ibid). Double labeling of cultured Schwann cells with NF-Fc and an antibody to gliomedin demonstrated that the extracellular domain of neurofascin only bound to cells expressing gliomedin (FIG. 2D indicating the binding of neurofascin to two gliomedin-expressing cells of the four cells present in the field). Furthermore, all the primary positive cDNA pools originating from the screen were found to contain gliomedin, indicating that we have isolated the main Schwann cell ligand for neurofascin.

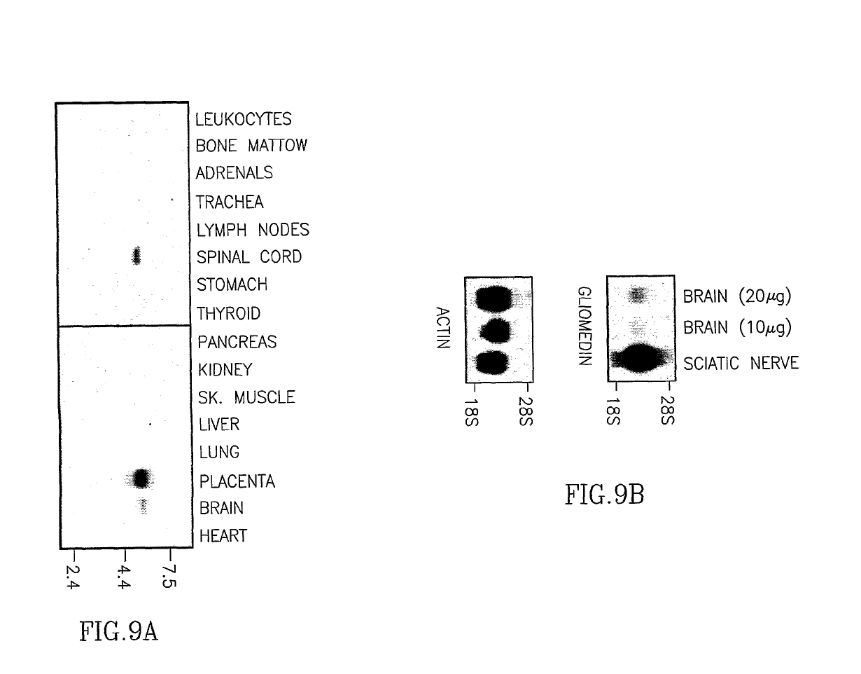

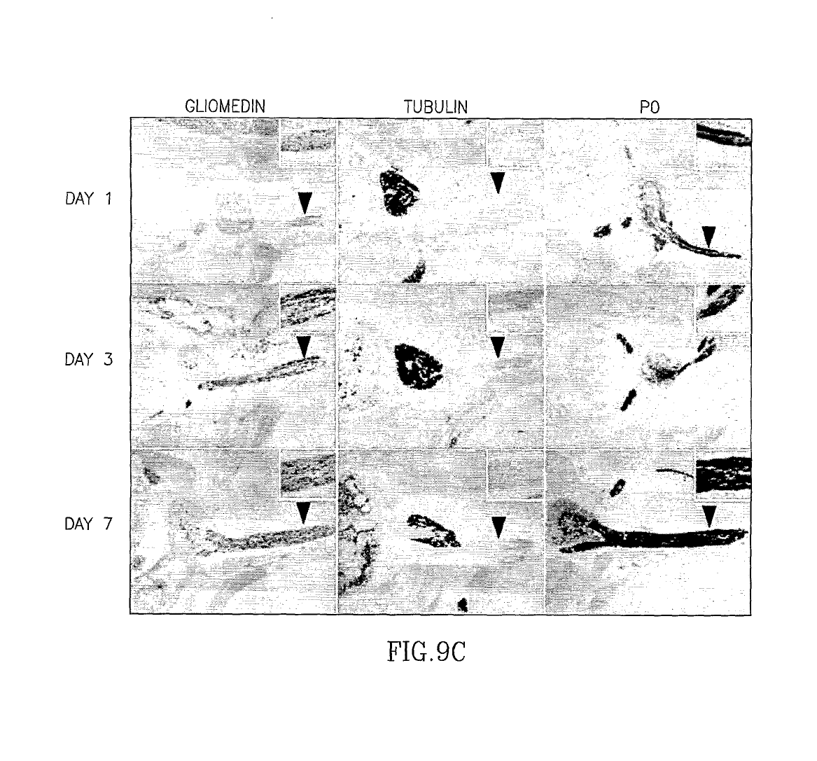

As demonstrated by Northern blot analysis, a single 5 kb gliomedin transcript was detected in human spinal cord, brain, placenta, and sciatic nerve but not in other tissues (FIG. 9). Direct comparison between the sciatic nerve and the brain showed that the gliomedin transcript is much more abundant in the PNS than in the CNS (FIG. 9B). 10 μg RNA isolated from the sciatic nerve were compared to the indicated amount of RNA prepared from the whole brain. Blots were hybridized with gliomedin or actin-specific probes as indicated. The location of 28S and 18S ribosomal RNA is shown on the right. In situ hybridization of rat sciatic nerve demonstrated a dramatic increase in the expression of gliomedin in myelinating Schwann cells during the first postnatal week, which corresponds to the initial period of active myelination in the PNS (FIG. 9C). Cross sections of DRGs connected to peripheral nerves prepared from 1, 3, or 7-day-old rats were hybridized to gliomedin, tubulin, or P0 probes. Tubulin and P0 served as markers for DRG neurons and myelinating Schwann cells, respectively.

### Example 3

### Gliomedin Interacts with Both Neurofascin and NrCAM

To characterize the interaction between gliomedin and cell adhesion molecules, we tested whether soluble Fc-fusion proteins containing the extracellular domain of various IgCAMs bind to cells expressing gliomedin. As depicted in FIG. 2E, soluble neurofascin (both isoforms) and NrCAM specifically bound to gliomedin-expressing COS7 cells. In contrast, no binding was detected when using Fc-fusion proteins containing the extracellular domain of other IgCAMs, such as L1 and contactin. Similar binding specificity was observed using isolated Schwann cells, or when a soluble gliomedin was used in binding experiments to COS7 cells expressing the different IgCAMs. Domain mapping analysis using soluble Fc-fusion proteins containing either the extracellular domain of gliomedin (ECD-Fc), its collagen repeat (COL-Fc), or the olfactomedin domain (OLF-Fc), demonstrated that the latter mediates its interaction with neurofascin and NrCAM (FIGS. 2F-2G). When ECD-Fc was used in binding experiments on frozen sections of sciatic nerves, it specifically labeled the nodes of Ranvier, where both neurofascin and NrCAM are located (FIG. 2H). Altogether, these experiments demonstrate that gliomedin binds specifically to the two IgCAMs that are found at the nodal axolemma and that the OLF domain of gliomedin mediates its interaction with neurofascin.

### Example 4

### Gliomedin is a Novel Component of PNS Nodes of Ranvier

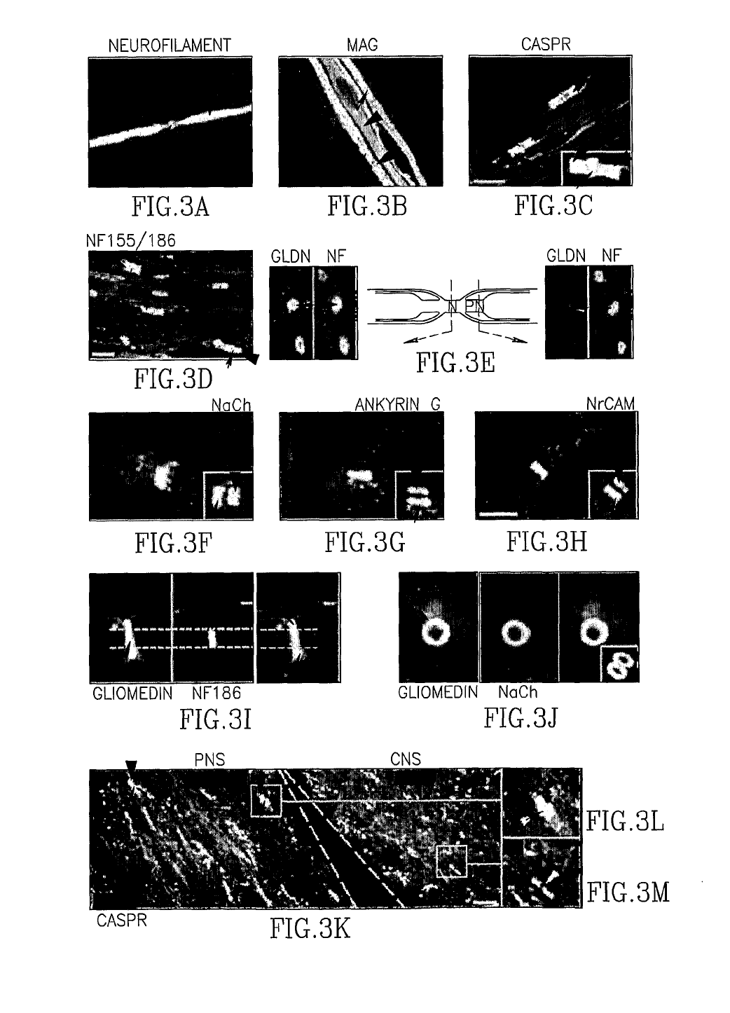

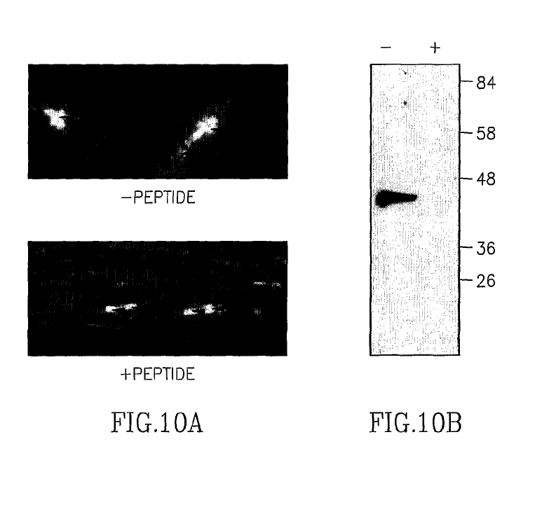

The localization of gliomedin in myelinated nerves was determined using polyclonal and monoclonal antibodies to this protein in combination with antibodies to various axonal or glial markers (FIGS. 3A-3M and FIG. 10). In sciatic nerve fibers from adult rats, gliomedin antibodies labeled short (1-2 μm-long) segments along the axon (FIG. 3A). Double labeling using antibodies to gliomedin and MAG (a marker for non-compact myelin) revealed that gliomedin was localized at the node of Ranvier, as identified by the flanking paranodal loops stained for MAG (FIG. 3B). Similarly, double immunolabeling with antibodies to gliomedin and Caspr, which marks the axoglial paranodal junction (Einheber, Peles et al., ibid), or an antibody that recognizes both isoforms of neurofascin and thus labels both nodes and paranodes (Schafer et al., 2004), demonstrated that gliomedin is found only in the nodes of Ranvier and not at the paranodes (FIGS. 3C-3E). Note that while both gliomedin and neurofascin are present in the nodes, only neurofascin is found at the adjacent paranodal region. The absence of gliomedin from the paranodal junction indicates that, in adult peripheral nerves, it is likely to interact with the 186-kDa isoforms of neurofascin that is found at the nodes rather than with the paranodal 155-kDa isoform. At the nodes, gliomedin was localized with known nodal proteins, including Na⁺ channels, ankyrin G, NrCAM and NF186 (FIG. 3F-J). Occasionally, gliomedin staining extended beyond the nodal axolemma (FIG. 3I), indicating that it may be expressed in the Schwann cell microvilli, which fill the nodal gap and contact the nodal axolemma in the PNS. In contrast, gliomedin was not detected at the nodes of Ranvier in the CNS as indicated in FIGS. 3K-3M. The figures show that while Caspr labeled the paranodes in both CNS and PNS, nodal labeling of gliomedin was only detected in the ventral roots and not in the spinal cord. Some weak, non-specific staining of gliomedin is also seen outside the nodes in the PNS.

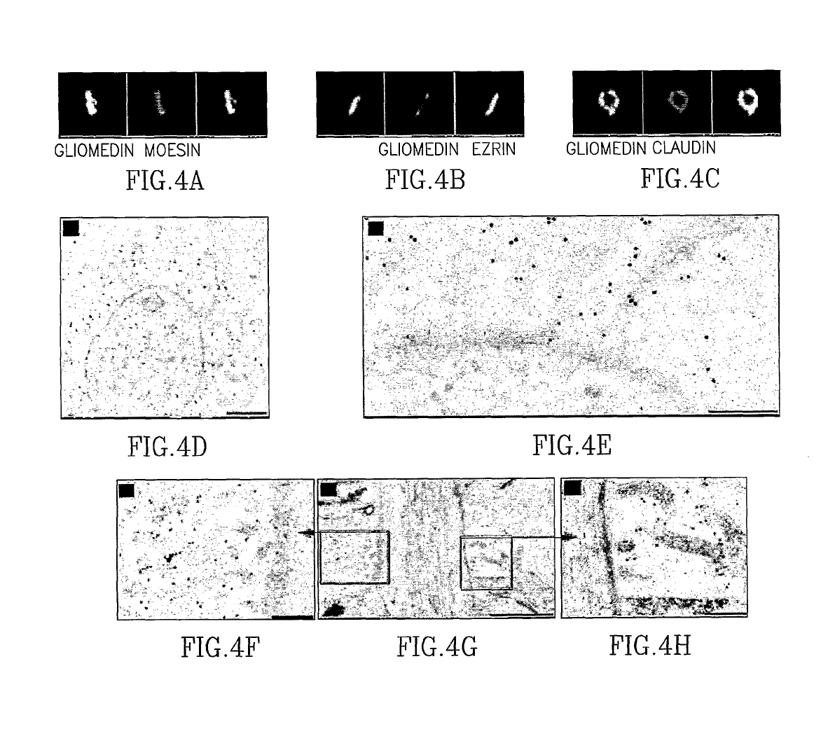

At PNS nodes, gliomedin is a component of the nodal Schwann cell microvilli: gliomedin is localized with ERM proteins (Ezrin, Radixin, Moesin) and claudin-2, all of which are concentrated in the Schwann cell microvilli (FIG. 4A). Furthermore, immunoelectron microscopy analysis of cross (FIG. 4D-E), or longitudinal (FIG. 4F-H) sections of adult rat sciatic nerve demonstrated that gliomedin is localized along the microvilli processes. Notably, strong labeling of the microvilli is detected, whereas no gold particles were detected in the axon. Furthermore, double labeling for gliomedin and ERM proteins revealed that gliomedin was always associated with a microvillar membrane. In summary, these results demonstrate that gliomedin is a novel glial component of the nodes of Ranvier in the PNS and suggest that it may play a role in mediating axoglial contact, as well as in maintaining the position of the microvilli towards the nodal axolemma.

### Example 5

### Developmental Expression of Gliomedin During Myelination

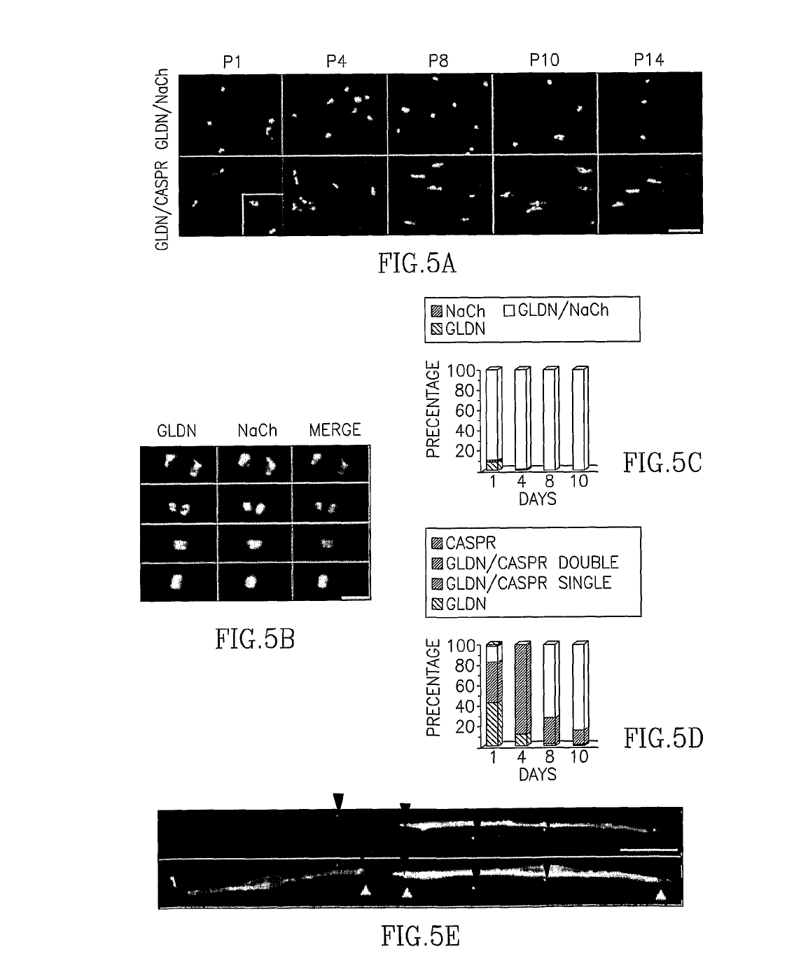

To examine when gliomedin accumulates at the node of Ranvier during the development of peripheral nerves, sections of rat sciatic nerves that had been collected from postnatal day 1 to 14 (P1-P14), were labeled with antibodies to gliomedin and to Na⁺ channels, or to Caspr (FIG. 5A). Gliomedin appeared at the node of Ranvier before Caspr accumulated at the paranodes (FIG. 5A), in accordance with previous studies demonstrating that Caspr accumulated at the paranodes shortly after ankyrin G and Na⁺ channels clustered at the nodes (Melendez-Vasquez et al., 2001, Proc Natl Acad Sci USA 98, 1235-1240). Subsequently, gliomedin-labeled nodes were flanked by one or two Caspr-positive paranodes (FIGS. 5A and D). Double labeling for gliomedin and Na⁺ channels demonstrated that clusters of the latter always co-localized with gliomedin (FIG. 5A). As previously described for Na⁺ channels, in the first postnatal days, antibodies to gliomedin labeled different intermediate forms of nodal maturation, ranging from distant binary nodes to closer binary and finally to the mature focal nodes (FIG. 5B). At P1, we occasionally detected gliomedin-positive sites that were negative for Na⁺ channels (FIG. 5C). Although it is not clear whether these early clusters indeed represent future nodes, they have been detected at the edges of MBP-labeled internodes, where Na⁺ clusters usually form. In contrast, we found that gliomedin co-localized with neurofascin at all stages of development of sciatic nerve. Triple immunolabeling of myelinating dorsal root ganglion explants with antibodies to gliomedin, MAG and MBP, demonstrated that the accumulation of gliomedin at edges of the internodes occurs with the transition between ensheathment (MAG⁺/MBP⁻) and the beginning of myelination (MAG⁺/MBP⁺) (FIG. 5E). As shown in FIG. 5E, clustering of gliomedin is detected in the cell on the right, which expresses both MAG and MBP, but is absent from the MAG positive cell on the left, which has not begun to express MBP. As found in developing nerves, neurofascin clusters were invariably associated with gliomedin during different stages of myelination in culture. These results demonstrate that gliomedin accumulates at the glial membrane that contacts the early forming node.

### Example 6

### Proper Localization of Gliomedin is Necessary for Node Formation

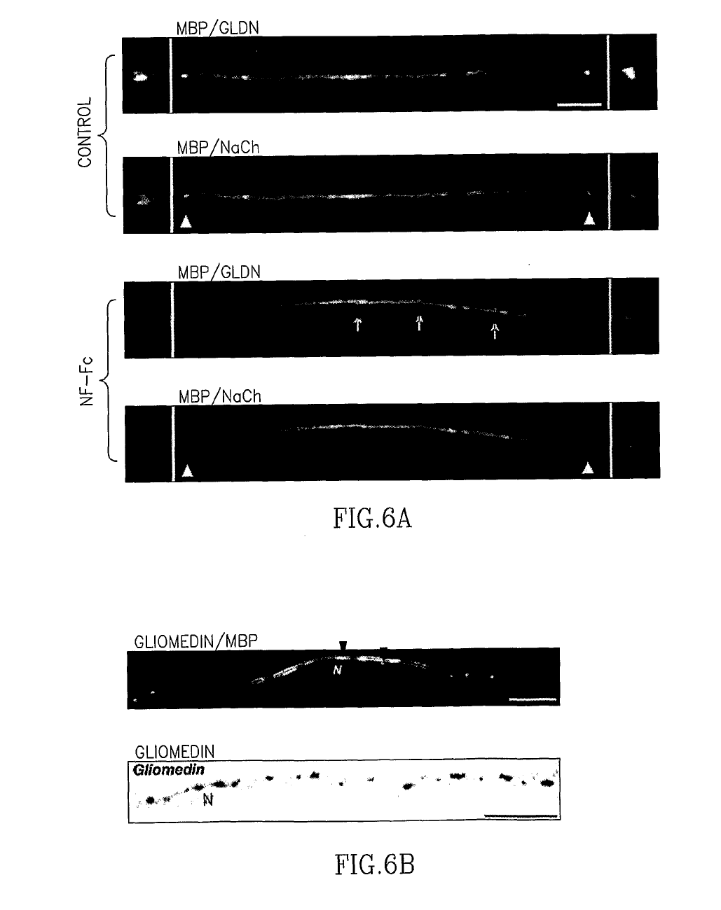

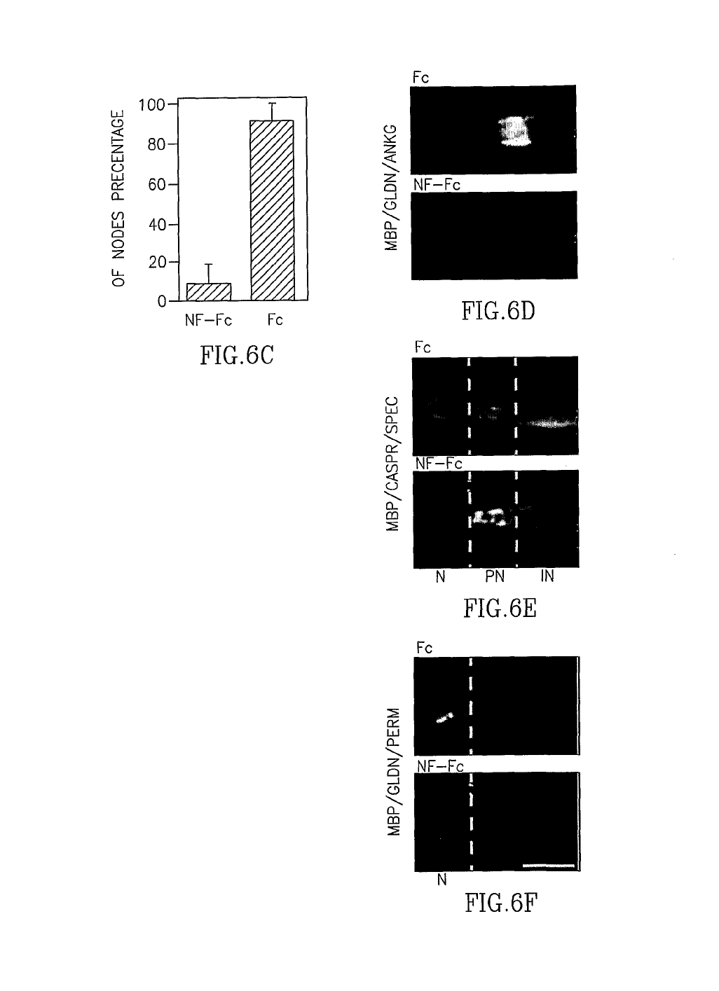

To elucidate the role of gliomedin in peripheral nerves, we first examined whether the addition of a soluble Fc-fusion protein containing the extracellular domain of its receptor (i.e., NF-Fc) to myelinating cultures affected node formation. DRG explants were grown in the presence of NF-Fc, or only the Fc region as control, for 12 days after the induction of myelination as described in the experimental procedures. Neither Fc-fusion protein had an affect on the ability of Schwann cells to myelinate, as determined by MBP expression (FIG. 6A). However, whereas gliomedin was located at both sides of MBP-labeled segments in control cultures, in the presence of NF-Fc, it was abnormally distributed along the internodes (FIG. 6A). Note that when some gliomedin was still present at the node it was associated with a small Na⁺ channel cluster. The addition of NF-Fc resulted in the clustering of gliomedin on the outer (abaxonal) membrane of the cell as indicated by aberrant distribution of gliomedin in FIG. 6B. This observation suggests that binding of NF-Fc caused the displacement of gliomedin on the cell surface. This notion was further supported by the observation that similar aggregates of gliomedin were detected on the surface of Schwann cells after 2 hours of incubation with NF-Fc. The absence of gliomedin from the nodes was accompanied by a loss of axonal clustering of Na⁺ channels, ankyrin G and PIV spectrin (FIGS. 6A and 6C-E). In contrast, phosphorylated ERM proteins were still present at nodal sites that lacked gliomedin (FIG. 6F), indicating that treatment of NF-Fc did not disrupt Schwann cell microvilli. Furthermore, when small and aberrant nodal clusters of Na⁺ channels were infrequently detected in the NF-Fc treated cultures, they were always found in proximity to some residual gliomedin staining (FIG. 6A, arrowhead in lower panel), demonstrating that the clustering of nodal components was invariably associated with the presence of gliomedin. Double labeling for Caspr and Na⁺ channels or βIV spectrin revealed the presence of sites that lacked nodal components but were still flanked by Caspr (see for example FIG. 6E), indicating that the effective nodal inhibition of NF-Fc was not secondary to paranodal abnormalities. This conclusion is further supported by previous studies demonstrating that the nodes are well formed in several paranodal mutants (Boyle, Peles et al., Neuron 30, 385-397, 2001), and the observation that gliomedin was localized at the nodes of Ranvier in Caspr null mice. In contrast to the robust effect of NF-Fc on the localization of gliomedin and node formation, we found that nodes were generated in myelinating DRG cultures treated with a soluble Fc-fusion protein containing the extracellular region of gliomedin. This result suggests that the soluble extracellular domain of gliomedin functions as the native protein and is sufficient for initiating node formation, an idea that we tested below.

### Example 7

### Gliomedin is Required for Nodal Clustering of Neurofascin and Na⁺ Channels

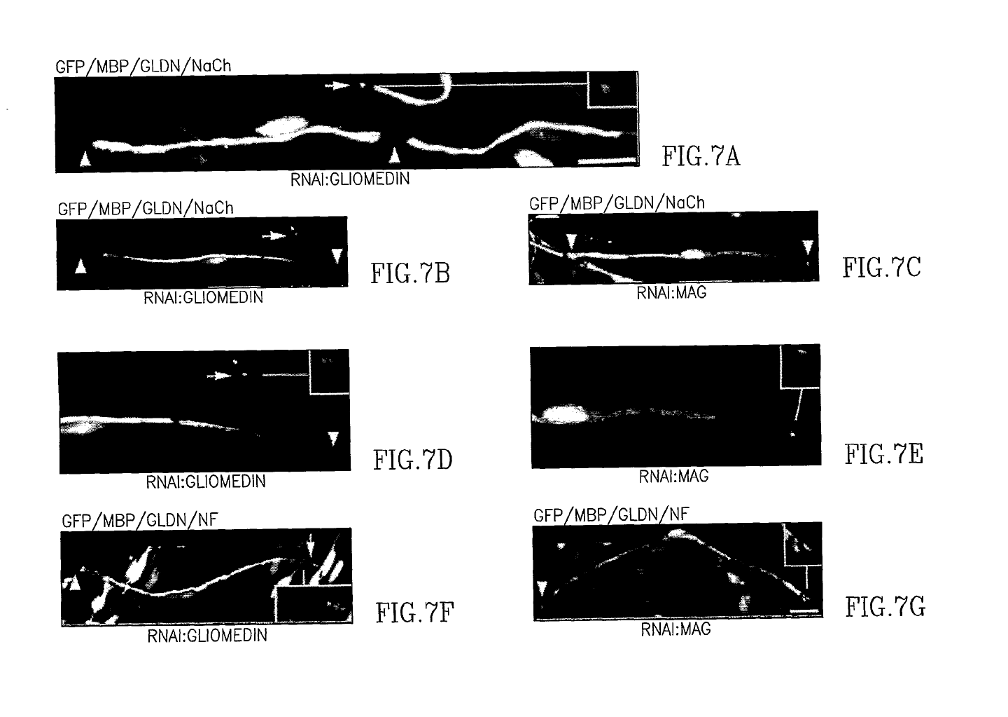

To further determine whether the expression of gliomedin in myelinating Schwann cells is essential for the clustering of nodal proteins at the axolemma, RNA interference (RNAi) were used to suppress its expression. Cultures of dissociated DRG neurons and Schwann cells were infected with retroviruses containing two different gliomedin-siRNA or a MAG-siRNA as control before the induction of myelination, as described in the Experimental Procedures. The infected cells were identified by the expression of GFP, which was included as a separate transcriptional unit in the viral vector used. While gliomedin clusters were present in 88% ( 283/322) of the MBP⁺GFP⁺ internodes expressing MAG-siRNA, they were present in only 26% ( 99/372) of the MBP⁺GFP⁺ internodes expressing gliomedin-siRNA, demonstrating the specificity of the siRNA used. Both gliomedin-siRNA and MAG-siRNA had no effect on the number of MBP-labeled internodes (FIG. 7), indicating that neither of these genes is required for myelination, as known in the art. As depicted in FIG. 7, suppression of gliomedin's expression by RNAi resulted in the abolishment of Na⁺ channels and neurofascin clustering at the axolemma. Importantly, Na⁺ channel clusters were never detected in more than 350 MBP⁺GFP⁺ internodes that lacked gliomedin, but were present at the edges such internodes that still expressed it. The inhibition of node formation was specific to gliomedin-RNAi as clusters of Na⁺ channels and neurofascin were present adjacent to gliomedin at the edges of myelin internodes of non infected cells (arrow in FIGS. 7A-B, 7D and 7F), or cells expressing MAG-RNAi (FIGS. 7C, 7E and 7G). Taken together with the inhibitory effect of NF-Fc described above, our findings demonstrate that the presence of gliomedin at the glial membrane opposing the nodes during development is necessary for the clustering of Na channel at the axolemma.

### Example 8

### Gliomedin Induces Nodal-Like Clustering in the Absence of Glial Cells

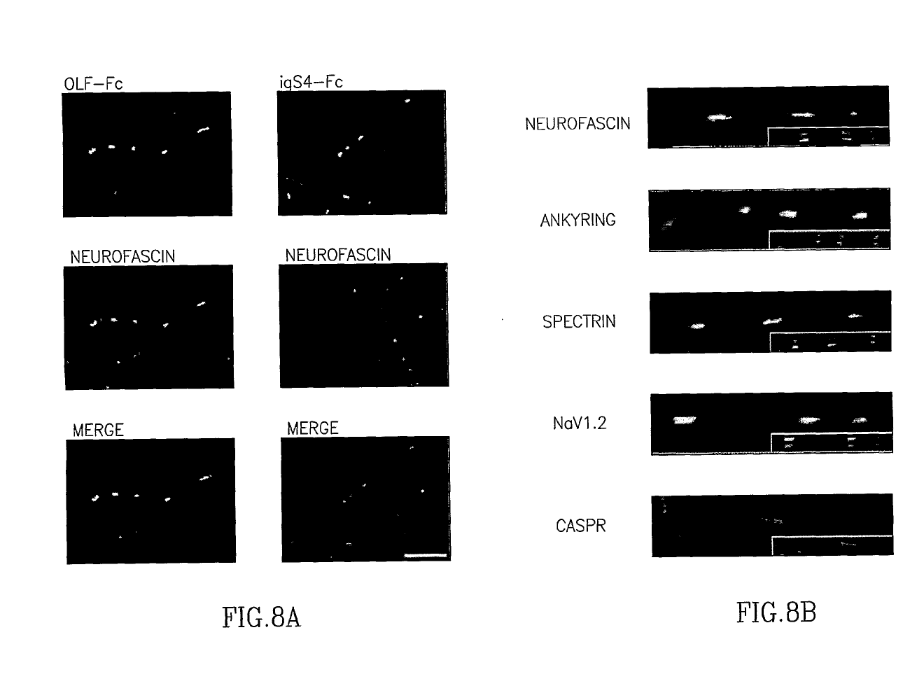

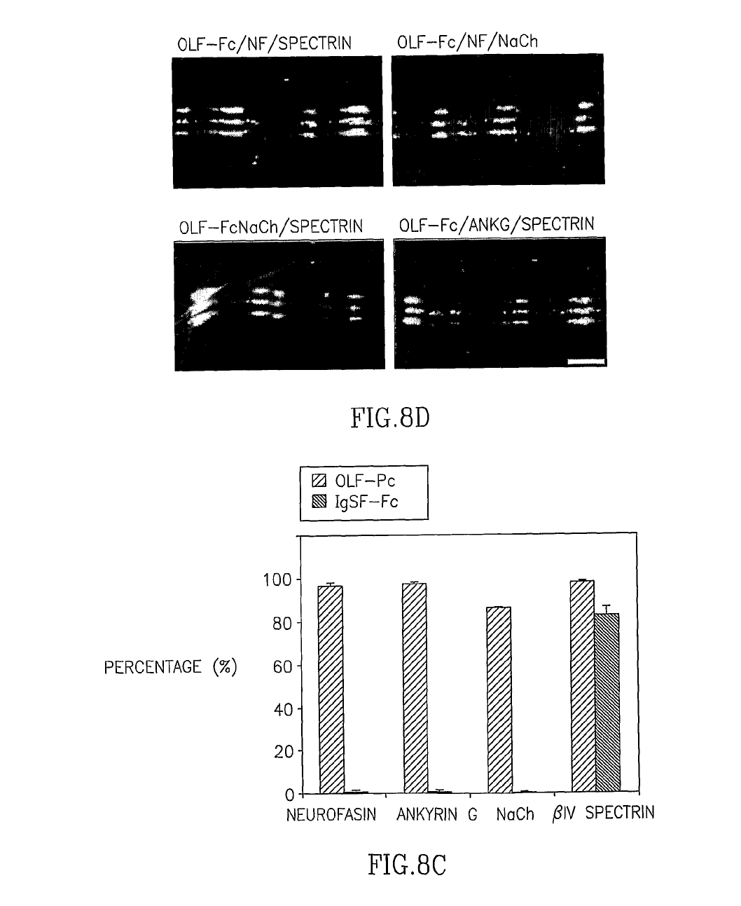

The interaction between gliomedin and the axonal IgCAMs may provide an inductive Schwann cell signal for the clustering of nodal components along the axolemma. To test this possibility, we examined whether the olfactomedin domain of gliomedin (OLF-Fc), which specifically binds neurofascin and NrCAM (FIG. 2), can induce clustering of Na⁺ channels in isolated DRG neurons. OLF-Fc was mixed with a Cy3-labeled antibody to human Fc and was allowed to bind purified DRG neurons for 30 minutes at 23° C. Further aggregation of the bound OLF-Fc by incubating the cultures at 37° C. for an additional 24 hours resulted in co-clustering of neurofascin (FIG. 8A), underscoring its role as the axonal receptor for gliomedin. Initial clustering of neurofascin was already detected after incubation of the cultures for 6 hours but was more pronounced 24-48 hours after the binding of OLF-Fc. In contrast, clustering of neurofascin was not detected using the extracellular domain of Igsf4c, an IgCAM that binds neurons (FIG. 8A). OLF-Fc treated DRG neurons were incubated for 48 hours at 37° C. in growth medium and then fixed and immunolabeled for the bound OLF-Fc (FIG. 8B). Remarkably, aggregation of OLF-Fc resulted in the recruitment of ankyrin G, βIV spectrin and Na⁺ channels to the neurofascin clusters (FIG. 8B). Aggregation of gliomedin induced the clustering of all the nodal components examined but not of Caspr or protein 4.1B. In contrast to the formation of these nodal-like clusters, OLF-Fc did not induce the clustering of other none-nodal axonal components, including transmembrane (Caspr) or cytoskeletal (protein 4.1b) proteins (FIG. 8B). IgSf4-Fc, which binds an unidentified neuronal receptor distinct from neurofascin or NrCAM, induced the clustering of PIV spectrin, but not of Na⁺ channels, ankyrin G and neurofascin (FIG. 8C), suggesting that although βIV spectrin is required for node formation, its clustering at the axonal membrane is not sufficient to induce the molecular assembly of the nodes. Quantification of these experiments revealed that 96% of the OLF-Fc clusters co-localized with neurofascin clusters, 97% with ankyrin G clusters, 86% contained Na⁺ channels clusters, and 98% of these sites co-localized with βIV spectrin clusters, indicating that all of the known nodal components examined simultaneously clustered by OLF-Fc (FIG. 8C). This conclusion was further supported by triple-labeling experiments demonstrating the co-clustering of neurofascin with βIV spectrin or Na⁺ channels, as well as of βIV spectrin with Na⁺ channels or ankyrin G at sites of OLF-Fc binding (FIG. 8D). DRG neurons were treated with OLF-Fc for 48 hours and then fixed and immunolabeled using a combination of antibodies as indicated in each panel. Co-clustering of neurofascin and spectrin, neurofascin and Na+ channels, Na+ channels and spectrin, as well as ankyrin G and spectrin at sites of gliomedin binding is noted (FIG. 8D). Interestingly, when used without a secondary antibody, OLF-Fc bound to DRG neurons but did not induce the formation of nodal-like clusters. These results demonstrate that the focal accumulation of gliomedin on the axonal surface is sufficient to induce the clustering of nodal components along the axon.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. An isolated polypeptide comprising the extracellular domain of human gliomedin, a fragment, an analog or a derivative thereof.

2. The isolated polypeptide of claim 1, comprising the entire extracellular domain as set forth in SEQ ID NO:18, a fragment, an analog or a derivative thereof.

3. The isolated polypeptide of claim 1, comprising the OLF domain as set forth in SEQ ID NO:19, a fragment, an analog or a derivative thereof.

4. The isolated polypeptide of claim 1, comprising the collagen repeat domain as set forth in SEQ ID NO:20, a fragment, an analog or a derivative thereof.

5. The isolated polypeptide of claim 1, comprising a chemical modification selected from the group consisting of: glycosylation, oxidation, permanent phosphorylation, reduction, myristylation, sulfation, acylation, acetylation, ADP-ribosylation, amidation, hydroxylation, iodination, methylation, and derivatization by blocking groups.

6. An isolated polynucleotide sequence encoding a polypeptide comprising the extracellular domain of human gliomedin, a fragment, an analog or a derivative thereof, wherein said polypeptide comprises the amino acid sequence selected from the group consisting of: SEQ ID NO:18, SEQ ID NO:19 and SEQ ID NO:20.

7. A pharmaceutical composition comprising as an active ingredient the polypeptide of any one of claims 1 through 5 and a pharmaceutically acceptable carrier.

8. The pharmaceutical composition of claim 7, wherein the pharmaceutically acceptable carrier is selected from the group consisting of: diluent, solubilizer, emulsifier, excipient, preservative, adjuvant and thickener.

9. An expression vector comprising the isolated polynucleotide of claim 6.

10. The expression vector of claim 9, comprising at least one regulatory element operatively linked to the polynucleotide, the at least one regulatory element being selected from the group consisting of: a promoter, an enhancer, a selectable gene, a signal peptide, a recombinase gene, a transcription factor gene and a reporter gene.

11. A pharmaceutical composition comprising as an active ingredient the vector of claim 9 and a pharmaceutically acceptable carrier.

12. A host cell expressing exogenous gliomedin or an active fragment thereof.

13. The host cell of claim 12, being transfected with an expression vector comprising a polynucleotide sequence encoding a polypeptide comprising the extracellular domain of human gliomedin, a fragment, an analog or a derivative thereof, wherein said polypeptide comprises the amino acid sequence selected from the group consisting of: SEQ ID NO:18, SEQ ID NO:19 and SEQ ID NO:20.

14. The host cell of claim 12, selected from the group consisting of: a somatic cell, a germ cell, a neuronal cell, pluripotent stem cell, nerve progenitor cell, Schwann cell, myelinating Schwann cell, glial cell and dorsal root ganglion neuron.

15. A monoclonal antibody capable of binding gliomedin or an extracellular fragment thereof.

16. The monoclonal antibody of claim 15, wherein the extracellular fragment comprises the amino acid sequence set forth by SEQ ID NO:18.

17. The monoclonal antibody of claim 15, selected from the group consisting of: humanized antibody, full-length antibody or an antibody fragment.

18. The monoclonal antibody of claim 17, wherein the antibody fragment is selected from the group consisting of: single chain antibody, Fab′, F(ab′)₂, and Fᵥ.

19. The monoclonal antibody of claim 15, further comprising a detectable label selected from the group consisting of: radionuclides, ultrasound contrast agents, MRI contrast agents, dyes, fluorescent compounds and paramagnetic metals.

20. The antibody of claim 15 being identical in function or activity to the antibody produced by cells deposited with the ATCC, deposition #______.

21. A double stranded siRNA molecule that down regulates expression of gliomedin via RNA interference.

22. The siRNA of claim 21, wherein:
(a) each strand of said siRNA molecule is independently about 15 to about 30 nucleotides in length; and
(b) one strand of said siRNA molecule comprises a nucleotide sequence having sufficient complementarity to an RNA of gliomedin.

23. The siRNA molecule of claim 21, comprising at least one modification selected from the group consisting of: 2′-sugar modification, at least one nucleic acid base modification, and at least one phosphate backbone modification.

24. The siRNA molecule of claim 21, comprising an oligonucleotide selected from SEQ ID NO:3, SEQ ID NO:4 and SEQ ID NO:5.

25. A method of treating neurological damage in a subject in need thereof, the methods comprising administering to the subject the pharmaceutical composition of any one of claims 7 and 11.

26. The method of claim 25, wherein the neurological damage is selected from the group consisting of: multiple sclerosis, axonal injury, lack of ion channel in axolemma, disordered axonal clustering of Na⁺ channels, damaged initial Schwann cell myelination, damaged initial nerve development and demyelination-associated disease.

27. The method of claim 26, wherein the demyelination-associated disease is selected from the group consisting of: diabetic neuropathy, Guillain-Bane Syndrome, chronic demyelinating disease, acute demyelinating polyneuropathy, human immunodeficiency viral demyelinating neuropathy, demyelination caused by trauma, inherited neuropathies, hereditary motor syndrome and sensory neuropathy.

28. A method of treating neurological damage in a subject in need thereof, the methods comprising transplanting into the subject the host cell of any one of claims 12 to 14.

29. The method of claim 28, wherein the transplanted host cells are autologous.

30. The method of claim 28, wherein the host cells being transplanted at or near at least one predetermined locus.

31. A method of diagnosing neurological damage, the method comprising
(a) administering to a subject in need thereof the monoclonal antibody of claim 15;
(b) detecting the antibody by imaging techniques; and,
optionally,
(c) evaluating the localization and/or amount of bound antibody and comparing said localization and/or amount with a localization and/or amount of bound antibody in a control healthy subject.

32. The method of claim 31, wherein the monoclonal antibody or fragment thereof is selected from the group consisting of: single chain antibody, Fab, Fab′, F(ab′)₂, and Fᵥ.

33. The method of claim 31, wherein the antibody is conjugated to a diagnostic agent.

34. The method of claim 33, wherein the diagnostic agent is selected from the group consisting of: radionuclides, ultrasound contrast agents, MRI contrast agents, dyes, fluorescent compounds and paramagnetic metals.

35. The method of claim 33, wherein the diagnostic agent is a radionuclide useful in positron emission, said radionuclide selected from the group consisting of F-18, Mn-51, Mn-52m, Fe-52, Co-55, Cu-62, Cu-64, Ga-68, As-72, Br-75, Br-76, Rb-82m, Sr-83, Y-86, Zr-89, Tc-94m, In-110, I-120 and I-124.

36. The method of claim 33, wherein said diagnostic agent is an MRI contrast agent comprising metals selected from the group consisting of gadolinium, manganese, iron, chromium, copper, cobalt, nickel, dysprosium, rhenium, europium, terbium, holmium and neodymium.

37. The method of claim 33, wherein said diagnostic agent is a radionuclide useful in gamma-ray detection and wherein said radionuclide is selected from the group consisting of Cr-51, Co-57, Co-58, Fe-59, Cu-67, Ga-67, Se-75, Ru-97, Tc-99m, In-111, In-114m, I-123, I-125, I-131, Yb-169, Hg-197 and Tl-201.

38. The method of claim 31, wherein said antibody is a humanized antibody.

39. The method of claim 31, further comprising administering to said subject a clearing agent and allowing said clearing agent to clear non-localized antibody.

40. The method of claim 39, wherein the clearing agent is an anti-idiotypic antibody or antigen-binding antibody fragment.

41. The method of claim 33, wherein the diagnostic agent is administered by a method selected from the group consisting of: intravenous bolus, intravenous perfusion, intraarterial, intrapleural, intraperitoneal, intrathecal and subcutaneous.

42. The method of claim 31, applied in conjunction with a method selected from: intraoperative probing, endoscopy and laparoscopy.

43. A method of down-regulating gliomedin expression in a cell, comprising contacting the cell with an siRNA molecules of claim 22, under conditions suitable for down-regulating gliomedin expression.

44. The method of claim 43, wherein the siRNA comprises at least one modification selected from the group consisting of: 2′-sugar modification, at least one nucleic acid base modification, and at least one phosphate backbone modification.

45. The method of claim 43, wherein the siRNA comprises an oligonucleotide selected from SEQ ID NO:3, SEQ ID NO:4 and SEQ ID NO:5.
